(19) United States
(12) Patent Application Publication
(10) Pub. No.: US 2004/0033251 A1
(43) Pub. Date: Feb. 19, 2004

(54) Active agent delivery system including a polyurethane, medical device, and method

(75) Inventors: Randall V. Sparer, Andover, MN (US); Christopher M. Hobot, Tonka Bay, MN (US); SuPing Lyu, Maple Grove, MN (US); Kiem Dang, Blaine, MN (US)

Correspondence Address: MUETING, RAASCH & GEBHARDT, P.A., P.O. BOX 581415, MINNEAPOLIS, MN, 55458, US

(73) Assignee: Medtronic, Inc., Minneapolis, MN

(21) Appl. No.: 10/640,823

(22) Filed: Aug. 13, 2003

Related U.S. Application Data
(60) Provisional application No. 60/403,478, filed on Aug. 13, 2002.

(51) Int. Cl. A61F 2/06; A61L 2/00; A61F 2/02

(52) U.S. Cl. 424/425; 427/2.24; 623/1.42

(57) ABSTRACT

The present invention provides active agent delivery systems for use in medical devices, wherein the active agent delivery systems include an active agent and a miscible polymer blend that includes a polyurethane and a second polymer, preferably one that has at least one Tg equal to or higher than all Tg's of the polyurethane.

### CROSS-REFERENCE TO RELATED APPLICATIONS

The present application claims priority to U.S. Provisional Patent Application Serial No. 60/403,478, filed on Aug. 13, 2002, which is incorporated herein by reference in its entirety.

### BACKGROUND OF THE INVENTION

A polymeric coating on a medical device may serve as a repository for delivery of an active agent (e.g., a therapeutic agent) to a subject. For many such applications, polymeric coatings must be as thin as possible. Polymeric materials for use in delivering an active agent may also be in various three-dimensional shapes.

Conventional active agent delivery systems suffer from limitations that include structural failure due to cracking and delamination from the device surface. Furthermore, they tend to be limited in terms of the range of active agents that can be used, the range of amounts of active agents that can be included within a delivery system, and the range of the rates at which the included active agents are delivered therefrom. This is frequently because many conventional systems include a single polymer.

Thus, there is a continuing need for active agent delivery systems with greater versatility and tunability.

### SUMMARY OF THE INVENTION

The present invention provides active agent delivery systems that have generally good versatility and tunability in controlling the delivery of active agents. Typically, such advantages result from the use of a blend of two or more miscible polymers. These delivery systems can be incorporated into medical devices, e.g., stents, stent grafts, anastomotic connectors, if desired.

The active agent delivery systems of the present invention typically include a blend of at least two miscible polymers, wherein at least one polymer (preferably one of the miscible polymers) is matched to the solubility of the active agent such that the delivery of the active agent preferably occurs predominantly under permeation control. In this context, “predominantly” with respect to permeation control means that at least 50%, preferably at least 75%, and more preferably at least 90%, of the total active agent load is delivered by permeation control.

Permeation control is typically important in delivering an active agent from systems in which the active agent passes through a miscible polymer blend having a “critical” dimension on a micron-scale level (i.e., the net diffusion path of no greater than about 1000 micrometers, although for shaped objects it can be up to about 10,000 microns). Furthermore, it is generally desirable to select polymers for a particular active agent that provide desirable mechanical properties without being detrimentally affected by nonuniform incorporation of the active agent.

In one preferred embodiment, the present invention provides an active agent delivery system that includes an active agent and a miscible polymer blend that includes a polyurethane and a second polymer, wherein the miscible polymer blend controls the delivery of the active agent. Preferably, the second polymer has at least one Tg equal to or higher than all Tg's of the polyurethane. Alternatively, the second polymer has at least one Tg lower than at least one Tg of the polyurethane.

More preferably, the second polymer is not a hydrophobic cellulose ester. Most preferably, the second polymer is selected from the group consisting of a polycarbonate, a polysulfone, a polyurethane, a polyphenylene oxide, a polyimide, a polyamide, a polyester, a polyether, a polyketone, a polyepoxide, a styrene-acrylonitrile copolymer, a polymethacrylate, a poly(methyl methacrylate), and combinations thereof. Preferably the active agent is not heparin.

In another preferred embodiment, the present invention provides an active agent delivery system that includes an active agent and a miscible polymer blend that includes a polyurethane and a second polymer, wherein: the second polymer is selected from the group consisting of a polycarbonate, a polysulfone, a polyurethane, a polyphenylene oxide, a polyimide, a polyamide, a polyester, a polyepoxide, a styrene-acrylonitrile copolymer, a polymethacrylate, a poly(methyl methacrylate), and combinations thereof; the active agent is hydrophobic and has a molecular weight of no greater than (i.e., less than or equal to) about 1200 grams per mole (g/mol); the active agent has a solubility parameter, the polyurethane has a hard segment solubility parameter and a soft segment solubility parameter, and the second polymer has at least one solubility parameter; the difference between the solubility parameter of the active agent and the polyurethane hard segment is no greater than about 10 J^{1/2}/cm^{3/2} (preferably, no greater than about 5 J^{1/2}/cm^{3/2}, and more preferably, no greater than about 3 J^{1/2}/cm^{3/2}), and/or the difference between the solubility parameter of the active agent and the polyurethane soft segment is no greater than about 10 J^{1/2}/cm^{3/2} (preferably, no greater than about 5 J^{1/2}/cm^{3/2}, and more preferably, no greater than about 3 J^{1/2}/cm^{3/2}), and the difference between the solubility parameter of the active agent and at least one solubility parameter of the second polymer (which, if the second polymer is a segmented polymer, is the solubility parameter of the hard and/or soft segment, for example) is no greater than about 10 J^{1/2}/cm^{3/2} (preferably, no greater than about 5 J^{1/2}/cm^{3/2}, and more preferably, no greater than about 3 J^{1/2}/cm^{3/2}); and the difference between the solubility parameter of the polyurethane hard segment and at least one solubility parameter of the second polymer (which, if the second polymer is a segmented polymer, is the solubility parameter of the hard and/or soft segment, for example) is no greater than about 5 J^{1/2}/cm^{3/2} (preferably, no greater than about 3 J^{1/2}/cm^{3/2}), and/or the difference between the solubility parameter of the polyurethane soft segment and at least one solubility parameter of the second polymer (which, if the second polymer is a segmented polymer, is the solubility parameter of the hard and/or soft segment, for example) is no greater than about 5 J^{1/2}/cm^{3/2} (preferably, no greater than about 3 J^{1/2}/cm^{3/2}).

In yet another preferred embodiment, an active agent delivery system includes: a miscible polymer blend that includes a first hydrophobic polymer selected from the group consisting of a poly(carbonate urethane) and a poly(ether urethane), and a second hydrophobic polymer selected from the group consisting of a polycarbonate, a poly(ether urethane), and a poly(carbonate urethane); wherein the first polymer has a hard phase Tg of about 20° C. to about 60° C. and the second polymer has at least one Tg of about 80° C. to about 150° C.; and an active agent incorporated in the miscible polymer blend, wherein the active agent is hydrophobic and has a molecular weight of no greater than about 1200 g/mol.

Hydrophobic miscible polymer blends can be used with hydrophilic active agents if the hydrophobic polymers have a solubility parameter preferably greater than 21 J^{1/2}/cm^{3/2} (more preferably, greater 25 J^{1/2}/cm^{3/2}). Typically, however, such blends are used in a reservoir system where the blend forms the cap coat overlying a base coat containing the hydrophilic active agent in a hydrophilic polymer. Thus, in still another preferred embodiment, an active agent reservoir delivery system includes: a base coat that includes a hydrophilic polymer and an active agent incorporated therein, wherein the active agent is hydrophilic and has a molecular weight of no greater than about 1200 g/mol; and a cap coat that includes a miscible polymer blend comprising a first hydrophobic poly(ether urethane) having a hard phase Tg of about 20° C. to about 60° C. and a second hydrophobic poly(ether urethane) having a hard phase Tg of about 80° C. to about 150° C.

As used herein, a “segmented polymer” is composed of multiple blocks, each of which can separate into the phase that is primarily composed of itself. As used herein, a “hard” segment or “hard” phase of a polymer is one that is either crystalline at use temperature or amorphous with a glass transition temperature above use temperature (i.e., glassy), and a “soft” segment or “soft” phase of a polymer is one that is amorphous with a glass transition temperature below use temperature (i.e., rubbery). Herein, a “segment” refers to the chemical formulation and “phase” refers to the morphology, which primarily includes the corresponding segment (e.g., hard segments form a hard phase), but can include some of the other segment (e.g., soft segments in a hard phase).

When referring to the solubility parameter of a segmented polymer, “segment” is used and when referring to Tg of a segmented polymer, “phase” is used. Thus, the solubility parameter, which is typically a calculated value for segmented polymers, refers to the hard and/or soft segment of an individual polymer molecule, whereas the Tg, which is typically a measured value, refers to the hard and/or soft phase of the bulk polymer.

The present invention also provides medical devices that include such active agent delivery systems.

In one preferred embodiment, a medical device is provided that includes: a substrate surface; a polymeric undercoat layer adhered to the substrate surface; and a polymeric top coat layer adhered to the polymeric undercoat layer; wherein the polymeric top coat layer includes an active agent incorporated within a miscible polymer blend that includes a polyurethane and a second polymer. Preferably, the second polymer has at least one Tg equal to or higher than all Tg's of the polyurethane. Alternatively, the second polymer has at least one Tg lower than at least one Tg of the polyurethane.

More preferably, the second polymer is selected from the group consisting of a polycarbonate, a polysulfone, a polyurethane, a polyphenylene oxide, a polyimide, a polyamide, a polyester, a polyepoxide, a styrene-acrylonitrile copolymer, a polymethacrylate, a poly(methyl methacrylate), and combinations thereof.

In another preferred embodiment, a stent is provided that includes: a substrate surface; a polymeric undercoat layer adhered to the substrate surface; and a polymeric top coat layer adhered to the undercoat layer; wherein the polymeric top coat layer includes an active agent incorporated within a miscible polymer blend that includes a polyurethane and a second polymer. Preferably, the second polymer has at least one Tg equal to or higher than all Tg's of the polyurethane. Alternatively, the second polymer has at least one Tg lower than at least one Tg of the polyurethane.

More preferably, the second polymer is selected from the group consisting of a polycarbonate, a polysulfone, a polyurethane, a polyphenylene oxide, a polyimide, a polyamide, a polyester, a polyepoxide, a styrene-acrylonitrile copolymer, a polymethacrylate, a poly(methyl methacrylate), and combinations thereof.

The present invention also provides methods for making an active agent delivery system and delivering an active agent to a subject. In one embodiment, a method of delivery includes: providing an active agent delivery system comprising an active agent and a miscible polymer blend comprising a polyurethane and a second polymer, wherein the miscible polymer blend controls the delivery of the active agent; and contacting the active agent delivery system with a bodily fluid, organ, or tissue of a subject. Preferably, the second polymer is not a hydrophobic cellulose ester.

In another embodiment, a method of forming an active agent delivery system includes: combining a polyurethane and a second polymer (preferably having at least one Tg equal to or higher than all Tg's of the polyurethane or, alternatively, having at least one Tg lower than at least one Tg of the polyurethane.) to form a miscible polymer blend; and combining at least one active agent with the miscible polymer blend such that the miscible polymer blend controls the delivery of the active agent. Preferably, the second polymer is not a hydrophobic cellulose ester.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1. Graph of the moduli of various poly(carbonate urethane) and poly(bis-phenol A carbonate) blends (PCU75D/PC blends) versus temperature. As the content of PC increased, the Tg of the individual polymers of the blends shifted closer together, indicating the PCU75D/PC blends were miscible.

FIG. 2. Graph of the cumulative release of dexamethasone from various PCU75D/PC blends versus the square root of time. The release rates were tuned by changing the amount of PCU75D of the blends.

FIG. 3. Graph of diffusion coefficient of dexamethasone in PCU75D/PC blends versus the composition of the blend. The diffusion coefficient increased as a function of the PCU75D content of the blends.

FIG. 4. Graph of the cumulative release of dexamethasone from various PELLETHANE 75D/PX blends (PX=a linear poly(bis-phenol A epoxide resin, numbers after PL in the legend indicating the weight percent (wt-%) of PELLETHANE 75D in the blends) versus the square root of time. The release rates were tuned by changing the amount of PELLETHANE 75D of the blends.

FIG. 5. DSC curves of PELLETHANE 75D/PHENOXY blends.

FIG. 6. Graph of the cumulative release of dexamethasone from various PCU75D/PCU55D blends (blends of two different poly(carbonate urethane)s, numbers after PCU75D in the legend indicating the wt-% of PCU75D in the blends). The release rates were tuned by changing the amount of PCU55D of the blends.

FIG. 7. Cumulative release of rosiglitasone maleate from various PELLETHANE 75D/PX blends (numbers after PL in the legend indicating the wt-% of PELLETHANE 75D in the blends). The release rates were tuned by changing the amount of PELLETHANE 75D of the blends.

FIG. 8. Cumulative percentage release of coumarin from PL75D/TP blend cap-coated shims.

FIG. 9. DSC curves of PL75D/TP blends that showed the miscibility between these two polymers.

### DETAILED DESCRIPTION OF ILLUSTRATIVE EMBODIMENTS

The present invention provides active agent delivery systems that include an active agent for delivery to a subject and a miscible polymer blend. The delivery systems can include a variety of polymers as long as at least two are miscible as defined herein. The active agent may be incorporated within the miscible polymer blend such that it is dissoluted from the blend, or the blend can initially function as a barrier to the environment through which the active agent passes.

Miscible polymer blends are advantageous because they can provide greater versatility and tunability for a greater range of active agents than can conventional systems that include immiscible mixtures or only a single polymer, for example. That is, using two or more polymers, at least two of which are miscible, can generally provide a more versatile active agent delivery system than a delivery system with only one of the polymers. A greater range of types of active agents can typically be used. A greater range of amounts of an active agent can typically be incorporated into and delivered from (preferably, predominantly under permeation control) the delivery systems of the present invention. A greater range of delivery rates for an active agent can typically be provided by the delivery systems of the present invention. At least in part, this is because of the use of a miscible polymer blend that includes at least two miscible polymers. It should be understood that, although the description herein refers to two polymers, the invention encompasses systems that include more than two polymers, as long as a miscible polymer blend is formed that includes at least two miscible polymers.

A miscible polymer blend of the present invention has a sufficient amount of at least two miscible polymers to form a continuous portion, which helps tune the rate of release of the active agent. Such a continuous portion (i.e., continuous phase) can be identified microscopically or by selective solvent etching. Preferably, the at least two miscible polymers form at least 50 percent by volume of a miscible polymer blend.

A miscible polymer blend can also optionally include a dispersed (i.e., discontinuous) immiscible portion. If both continuous and dispersed portions are present, the active agent can be incorporated within either portion. Preferably, the active agent is loaded into the continuous portion to provide delivery of the active agent predominantly under permeation control. To load the active agent, the solubility parameters of the active agent and the portion of the miscible polymer blend a majority of the active agent is loaded into are matched matched (typically to within no greater than about 10 J^{1/2}/cm^{3/2}, preferably, no greater than about 5 J^{1/2}/cm^{3/2}, and more preferably, no greater than about 3 J^{1/2}/cm^{3/2}). The continuous phase controls the release of the active agent regardless of where the active agent is loaded.

A miscible polymer blend, as used herein, encompasses a number of completely miscible blends of two or more polymers as well as partially miscible blends of two or more polymers. A completely miscible polymer blend will ideally have a single glass transition temperature (Tg), preferably one in each phase (typically a hard phase and a soft phase) for segmented polymers, due to mixing at the molecular level over the entire concentration range. Partially miscible polymer blends may have multiple Tg's, which can be in one or both of the hard phase and the soft phase for segmented polymers, because mixing at the molecular level is limited to only parts of the entire concentration range. These partially miscible blends are included within the scope of the term “miscible polymer blend” as long as the absolute value of the difference in at least one Tg (Tg_{polymer 1}-Tg_{polymer 2}) (preferably, the highest Tg's) for each of at least two polymers within the blend is reduced by the act of blending. Tg's can be determined by measuring the mechanical properties, thermal properties, electric properties, etc. as a function of temperature.

A miscible polymer blend can also be determined based on its optical properties. A completely miscible blend forms a stable and homogeneous domain that is transparent, whereas an immiscible blend forms a heterogeneous domain that scatters light and visually appears turbid unless the components have identical refractive indices. Additionally, a phase-separated structure of immiscible blends can be directly observed with microscopy. A simple method used in the present invention to check the miscibility involves mixing the polymers and forming a thin film of about 10 micrometers to about 50 micrometers thick. If such a film is generally as clear and transparent as the least clear and transparent film of the same thickness of the individual polymers prior to blending, then the polymers are completely miscible.

Miscibility between polymers depends on the interactions between them and their molecular structures and molecular weights. The interaction between polymers can be characterized by the so-called Flory-Huggins parameter (χ). When χ is close to zero (0) or even is negative, the polymers are very likely miscible. Theoretically, χ can be estimated from the solubility parameters of the polymers, i.e., χ is proportional to the squared difference between them. Therefore, the miscibility of polymers can be approximately predicted. For example, the closer the solubility parameters of the two polymers are the higher the possibility that the two polymers are miscible. Miscibility between polymers tends to decrease as their molecular weights increases.

Thus, in addition to the experimental determinations, the miscibility between polymers can be predicted simply based on the Flory-Huggins interaction parameters, or even more simply, based the solubility parameters of the components. However, because of the molecular weight effect, close solubility parameters do not necessarily guarantee miscibility.

It should be understood that a mixture of polymers needs only to meet one of the definitions provided herein to be miscible. Furthermore, a mixture of polymers may become a miscible blend upon incorporation of an active agent. As used herein, a “hard” phase of a blend includes primarily a segmented polymer's hard segment and optionally at least part of a second polymer blended therein. Similarly, a “soft” phase of a blend includes predominantly a segmented polymer's soft segment and optionally at least part of a second polymer blended therein. Preferably, miscible blends of polymers of the present invention include blends of segmented polymers' soft segments.

The types and amounts of polymers and active agents are typically selected to form a system having a preselected dissolution time (or rate) through a preselected critical dimension of the miscible polymer blend. Glass transition temperatures and solubility parameters can be used in guiding one of skill in the art to select an appropriate combination of components in an active agent delivery system, whether the active agent is incorporated into the miscible polymer blend or not. Solubility parameters are generally useful for determining the miscibility of the polymers and matching the solubility of the active agent to that of the miscible polymer blend. Glass transition temperatures are generally useful for determining miscibility of polymers and tuning the dissolution time (or rate) of the active agent. These concepts are discussed in greater detail below.

A miscible polymer blend can be used in combination with an active agent in the delivery systems of the present invention in a variety of formats as long as the miscible polymer blend controls the delivery of the active agent.

In one embodiment, a miscible polymer blend has an active agent incorporated therein. Preferably, such an active agent is dissoluted predominantly under permeation control, which requires at least some solubility of the active agent in the continuous portion (i.e., the miscible portion) of the polymer blend, whether the majority of the active agent is loaded in the continuous portion or not. Dispersions are acceptable as long as little or no porosity channeling occurs during dissolution of the active agent and the size of the dispersed domains is much smaller than the critical dimension of the blends, and the physical properties are generally uniform throughout the composition for desirable mechanical performance. This embodiment is often referred to as a “matrix” system.

In another embodiment, a miscible polymer blend initially provides a barrier to permeation of an active agent. This embodiment is often referred to as a “reservoir” system. A reservoir system can be in many formats with two or more layers. For example, a miscible polymer blend can form an outer layer over an inner layer of another material (referred to herein as the inner matrix material). In another example, a reservoir system can be in the form of a core-shell, wherein the miscible polymer blend forms the shell around the core matrix (i.e., the inner matrix material). At least initially upon formation, the miscible polymer blend in the shell or outer layer could be substantially free of active agent. Subsequently, the active agent permeates from the inner matrix and through the miscible polymer blend for delivery to the subject. The inner matrix material can include a wide variety of conventional materials used in the delivery of active agents. These include, for example, an organic polymer such as those described herein for use in the miscible polymer blends, or a wax, or a different miscible polymer blend. Alternatively, the inner matrix material can be the active agent itself.

For a reservoir system, the release rate of the active agent can be tuned with selection of the material of the outer layer. The inner matrix can include an immiscible mixture of polymers or it can be a homopolymer if the outer layer is a miscible blend of polymers. A reservoir system is prepared in Example 5.

As with matrix systems, an active agent in a reservoir system is preferably dissoluted predominantly under permeation control through the miscible polymer blend of the barrier layer (i.e., the barrier polymer blend), which requires at least some solubility of the active agent in the barrier polymer blend. Again, dispersions are acceptable as long as little or no porosity channeling occurs in the barrier polymer blend during dissolution of the active agent and the size of the dispersed domains is much smaller than the critical dimension of the blends, and the physical properties are generally uniform throughout the barrier polymer blend for desirable mechanical performance. Although these considerations may also be desirable for the inner matrix, they are not necessary requirements.

Typically, the amount of active agent within an active agent delivery system of the present invention is determined by the amount to be delivered and the time period over which it is to be delivered. Other factors can also contribute to the level of active agent present, including, for example, the ability of the composition to form a uniform film on a substrate.

Preferably, for a matrix system, an active agent is present within (i.e., incorporated within) a miscible polymer blend in an amount of at least about 0.1 weight percent (wt-%), more preferably, at least about 1 wt-%, and even more preferably, at least about 5 wt-%, based on the total weight of the miscible polymer blend and the active agent. Preferably, for a matrix system, an active agent is present within a miscible polymer blend in an amount of no greater than about 80 wt-%, more preferably, no greater than about 50 wt-%, and most preferably, no greater than about 30 wt-%, based on the total weight of the miscible polymer blend and the active agent. Typically and preferably, the amount of active agent will be at or below its solubility limit in the miscible polymer blend.

Preferably, for a reservoir system, an active agent is present within an inner matrix (e.g., a base layer) in an amount of at least about 0.1 wt-%, more preferably, at least about 10 wt-%, and even more preferably, at least about 25 wt-%, based on the total weight of the inner matrix (including the active agent). Preferably, for a reservoir system, an active agent is present within an inner matrix in an amount up to 100 wt-%, and more preferably, no greater than about 80 wt-%, based on the total weight of the inner matrix (including the active agent).

In the active agent delivery systems of the present invention, an active agent is dissolutable through a miscible polymer blend. Dissolution is preferably controlled predominantly by permeation of the active agent through the miscible polymer blend. That is, the active agent initially dissolves into the miscible polymer blend and then diffuses through the miscible polymer blend predominantly under permeation control. Thus, release of the active agent is typically not controlled by porosity in the miscible polymer blends or by polymer degradation. Thus, as stated above, for certain preferred embodiments, the active agent is at or below the solubility limit of the miscible polymer blend. Although not wishing to be bound by theory, it is believed that because of this mechanism the active agent delivery systems of the present invention have a significant level of tunability.

If the active agent exceeds the solubility of the miscible polymer blend and the amount of insoluble active agent exceeds the percolation limit, then the active agent could be dissoluted predominantly through a porosity mechanism. In addition, if the largest dimension of the active agent insoluble phase (e.g., particles or aggregates of particles) is on the same order as the critical dimension of the miscible polymer blend, then the active agent could be dissoluted predominantly through a porosity mechanism. Dissolution by porosity control is typically undesirable because it does not provide effective predictability and controllability.

Because the active agent delivery systems of the present invention preferably have a critical dimension on the micron-scale level, it can be difficult to include a sufficient amount of active agent and avoid delivery by a porosity mechanism. Thus, the solubility parameters of the active agent and at least one polymer of the miscible polymer blend are matched to maximize the level of loading while decreasing the tendency for delivery by a porosity mechanism.

One can determine if there is a permeation-controlled release mechanism by examining a dissolution profile of the amount of active agent released versus time (t). For permeation-controlled release from a matrix system, the profile is directly proportional to t^{1/2}. For permeation-controlled release from a reservoir system, the profile is directly proportional to t. Alternatively, under sink conditions (i.e., conditions under which there are no rate-limiting barriers between the polymer blend and the media into which the active agent is dissoluted), porosity-controlled dissolution could result in a burst effect (i.e., an initial very rapid release of active agent).

The active agent delivery systems of the present invention, whether in the form of a matrix system or a reservoir system, for example, without limitation, can be in the form of coatings on substrates (e.g., open or closed cell foams, woven or nonwoven materials), films (which can be free-standing as in a patch, for example), shaped objects (e.g., microspheres, beads, rods, fibers, or other shaped objects), wound packing materials, etc. As used herein, an “active agent” is one that produces a local or systemic effect in a subject (e.g., an animal). Typically, it is a pharmacologically active substance. The term is used to encompass any substance intended for use in the diagnosis, cure, mitigation, treatment, or prevention of disease or in the enhancement of desirable physical or mental development and conditions in a subject. The term “subject” used herein is taken to include humans, sheep, horses, cattle, pigs, dogs, cats, rats, mice, birds, reptiles, fish, insects, arachnids, protists (e.g., protozoa), and prokaryotic bacteria. Preferably, the subject is a human or other mammal.

Active agents can be synthetic or naturally occurring and include, without limitation, organic and inorganic chemical agents, polypeptides (which is used herein to encompass a polymer of L- or D-amino acids of any length including peptides, oligopeptides, proteins, enzymes, hormones, etc.), polynucleotides (which is used herein to encompass a polymer of nucleic acids of any length including oligonucleotides, single- and double-stranded DNA, single- and double-stranded RNA, DNA/RNA chimeras, etc.), saccharides (e.g., mono-, di-, poly-saccharides, and mucopolysaccharides), vitamins, viral agents, and other living material, radionuclides, and the like. Examples include antithrombogenic and anticoagulant agents such as heparin, coumadin, coumarin, protamine, and hirudin; antimicrobial agents such as antibiotics; antineoplastic agents and anti-proliferative agents such as etoposide, podophylotoxin; antiplatelet agents including aspirin and dipyridamole; antimitotics (cytotoxic agents) and antimetabolites such as methotrexate, colchicine, azathioprine, vincristine, vinblastine, fluorouracil, adriamycin, and mutamycinnucleic acids; antidiabetic such as rosiglitazone maleate; and anti-inflammatory agents. Anti-inflammatory agents for use in the present invention include glucocorticoids, their salts, and derivatives thereof, such as cortisol, cortisone, fludrocortisone, Prednisone, Prednisolone, 6α-methylprednisolone, triamcinolone, betamethasone, dexamethasone, beclomethasone, aclomethasone, amcinonide, clebethasol, and clocortolone. Preferably, the active agent is not heparin.

For preferred active agent delivery systems of the present invention, the active agent is typically matched to the solubility of the miscible portion of the polymer blend. For the present invention, at least one polymer of the polymer blend is a polyurethane, which is typically hydrophobic. For certain embodiments, the preferred active agents are hydrophobic and for certain other embodiments, the preferred active agents are hydrophilic. Preferably, if the active agent is hydrophobic, then at least one of the miscible polymers is hydrophobic, and if the active agent is hydrophilic, then at least one of the miscible polymers is hydrophilic. However, this is not necessarily required, and it may be undesirable to have a hydrophilic polymer in a delivery system for a low molecular weight hydrophilic active agent because of the potential for swelling of the polymers by water and the loss of controlled delivery of the active agent.

As used herein, in this context (in the context of the polymer of the blend), the term “hydrophobic” refers to a material that will not increase in volume by more than 10% or in weight by more than 10%, whichever comes first, when swollen by water at body temperature (i.e., about 37° C.). In contrast, the term “hydrophilic” refers to a material that will increase in volume by at least 10% or in weight by at least 10%, whichever comes first, when swollen by water at body temperature (i.e., about 37° C.).

As used herein, in this context (in the context of the active agent), the term “hydrophobic” refers to an active agent that has a solubility in water at room temperature (i.e., about 25° C.) of no more than (i.e., less than or equal to) 200 micrograms per milliliter. In contrast, the term “hydrophilic” refers to an active agent that has a solubility in water of more than 200 micrograms per milliliter.

For delivery systems in which the active agent is hydrophobic, regardless of the molecular weight, polymers are typically selected such that the molar average solubility parameter of the miscible polymer blend is no greater than 28 J^{1/2}/cm^{3/2} (preferably, no greater than 25 J^{1/2}/cm^{3/2}). For delivery systems in which the active agent is hydrophilic, regardless of the molecular weight, polymers are typically selected such that the molar average solubility parameter of the miscible polymer blend is greater than 21 J^{1/2}/cm^{3/2} (preferably, greater than 25 J^{1/2}/cm^{3/2}). Herein “molar average solubility parameter” means the average of the solubility parameters of the blend components that are miscible with each other and that form the continuous portion of the miscible polymer blend. These are weighted by their molar percentage in the blend, without the active agent incorporated into the polymer blend.

As the size of the active agent gets sufficiently large, diffusion through the polymer is affected. Thus, active agents can be categorized based on molecular weights and polymers can be selected depending on the range of molecular weights of the active agents.

For preferred active agent delivery systems of the present invention, the active agent has a molecular weight of no greater than about 1200 g/mol. For even more preferred embodiments, active agents of a molecular weight no greater than about 800 g/mol are desired.

Of the active agents listed above, those that are hydrophobic and have a molecular weight of no greater than about 1200 g/mol are particularly preferred.

As stated above, the types and amounts of polymers and active agents are typically selected to form a system having a preselected dissolution time (t) through a preselected critical dimension (x) of the miscible polymer blend. This involves selecting at least two polymers to provide a target diffusivity, which is directly proportional to the critical dimension squared divided by the time (x²/t), for a given active agent.

The diffusivity can be easily measured by dissolution analysis using the following equation (see, for example, Kinam Park edited, Controlled Drug Delivery: Challenges and Strategies, American Chemical Society, Washington, D.C., 1997):

$$D = {( \frac{{M}_{t}}{4 {M}_{∞}} )}^{2} \cdot \frac{π {x}^{2}}{t}$$

wherein D=diffusion coefficient; Mₜ=cumulative release; M∞=total loading of active agent; x=the critical dimension (e.g., thickness of the film); and t=the dissolution time. This equation is valid during dissolution of up to 60 percent by weight of the initial load of the active agent. Also, blend samples should be in the form of a film.

In refining the selection of the polymers for the desired active agent, the desired dissolution time (or rate), and the desired critical dimension, the parameters that can be considered when selecting the polymers for the desired active agent include glass transition temperatures of the polymers, solubility parameters of the polymers, and solubility parameters of the active agents. These can be used in guiding one of skill in the art to select an appropriate combination of components in an active agent delivery system, whether the active agent is incorporated into the miscible polymer blend or not.

For enhancing the tunability of a permeation-controlled delivery system, for example, preferably the polymers are selected such that the difference between at least one Tg of at least two of the polymers of the blend is sufficient to provide the target diffusivity. The target diffusivity is determined by the preselected dissolution time (t) for delivery and the preselected critical dimension (x) of the polymer composition and is directly proportional to x²/t.

For enhancing the versatility of a permeation-controlled delivery system, for example, preferably the polymers are selected such that at least one of the following relationships is true: (1) the difference between the solubility parameter of the active agent and at least one solubility parameter of at least one polymer is no greater than about 10 J^{1/2}/cm^{3/2} (preferably, no greater than about 5 J^{1/2}/cm^{3/2}, and more preferably, no greater than about 3 J^{1/2}/cm^{3/2}); and (2) the difference between at least one solubility parameter of each of at least two polymers is no greater than about 5 J^{1/2}/cm^{3/2} (preferably, no greater than about 3 J^{1/2}/cm^{3/2}). More preferably, both relationships are true. Most preferably, both relationships are true for all polymers of the blend.

Typically, a compound has only one solubility parameter, although certain polymers, such as segmented copolymers and block copolymers, for example, can have more than one solubility parameter. Solubility parameters can be measured or they are calculated using an average of the values calculated using the Hoy Method and the Hoftyzer-van Krevelen Method (chemical group contribution methods), as disclosed in D. W. van Krevelen, Properties of Polymers, 3^{rd} Edition, Elsevier, Amsterdam. To calculate these values, the volume of each chemical is needed, which can be calculated using the Fedors Method, disclosed in the same reference.

Solubility parameters can also be calculated with computer simulations, for example, molecular dynamics simulation and Monte Carlo simulation. Specifically, the molecular dynamics simulation can be conducted with Accelrys Materials Studio, Accelrys Inc., San Diego, Calif. The computer simulations can be used to directly calculate the Flory-Huggins parameter.

A miscible polymer blend of the present invention includes a polyurethane, which can be a homopolymer or copolymer. Herein, a “copolymer” includes two or more different repeat units, thereby encompassing terpolymers, tetrapolymers, and the like. The polyurethane is typically hydrophobic. As used herein in this context (in the context of the polymer matrix), the term “hydrophobic” refers to a material that will not increase in volume by more than 10% or in weight by more than 10%, whichever comes first, when swollen by water at body temperature (i.e., about 37° C.).

A polyurethane is preferably present in the miscible polymer blend in an amount of at least about 0.1 wt-%, and more preferably up to about 99.9 wt-%, based on the total weight of the blend, depending on the active agent and specific choice of polymers.

A particularly preferred polyurethane has a Shore durometer hardness of at least about 50A, more preferably at least about 55D, and most preferably at least about 70D. A particularly preferred polyurethane has a Shore durometer hardness of no greater than about 90D, more preferably no greater than about 85D, and most preferably no greater than about 80D. The hardness numbers are derived from the Shore scale, with the A scale being used for softer and the D scale being used for harder materials.

Suitable polyurethanes are available from a variety of sources such as Thermedics, Inc. (Woburn, Mass.), including polymers marketed under the tradenames TECOPLAST, TECOTHANE, CARBOTHANE, and TECOFLEX. Other preferred polymers include the PELLETHANE and ISOPLAST series available from Dow Chemical Co. (Midland, Mich.), especially PELLETHANE 75D; ELASTHANE, PURSIL, CARBOSIL, BIONATE, and BIOSPAN, available from the Polymer Technology Group, Inc. (Berkeley, Calif.); ESTANE, available from Noveon, Inc. (Cleveland, Ohio); ELAST-EON, available from AorTech Biomaterials (Sidney, Australia); and TEXIN, available from Bayer (Pittsburg, Pa.).

Examples of such polyurethanes include poly(carbonate urethane), poly(ether urethane), poly(ester urethane), poly(siloxane urethane), poly(hydrocarbon urethane), such as those exemplified in U.S. Pat. No. 4,873,308, sulfur-containing polyurethanes, such as those exemplified in U.S. Pat. Nos. 6,149,678, 6,111,052, 5,986,034, end-group modified polyurethanes, such as those commercially available from Polymer Technology Group, Inc., under the trade designation SME, or combinations thereof. Additionally, the polyurethanes may be derived from isocyanates including aromatic and/or aliphatic groups. A particularly preferred polyurethane is a poly(carbonate urethane) or a poly(ether urethane).

Preferably, higher molecular weights of polymers are desirable for better mechanical properties; however, the molecular weights should not be so high such that the polymer is not soluble in a processing solvent for preferred solvent-coating techniques or not miscible with the other polymer(s) in the blend. A preferred polyurethane has a number average molecular weight of at least about 20,000 g/mol, and more preferably at least about 80,000 g/mol. A preferred polyurethane has a number average molecular weight of no greater than about 1,000,000 g/mol, and more preferably no greater than about 300,000 g/mol.

A miscible polymer blend of the present invention includes at least a second polymer. The second polymer can have at least one Tg equal to or higher than any one Tg of the polyurethane. Alternatively, the second polymer can have at least one Tg equal to or lower than any one Tg of the polyurethane. Preferably, the second polymer has at least one Tg higher than all Tg's of the polyurethane. This includes a wide variety of polymers such that the act of blending this second polymer with the polyurethane, the absolute value of the difference in at least one Tg (Tg_{polymer 1}-Tg_{polymer 2}) for each of at least two polymers within the blend is reduced by the act of blending.

Alternatively, the second polymer has at least one Tg lower than at least one Tg of the polyurethane.

This second polymer may also be a homopolymer or a copolymer. A second polymer is preferably present in the miscible polymer blend in an amount of at least about 0.1 wt-%, and more preferably up to about 99.9 wt-%, based on the total weight of the blend, depending on the active agent and specific choice of polymers.

For embodiments in which the second polymer has a Tg higher than all Tg's of the polyurethane, the second polymer is preferably selected from the group consisting of a polycarbonate, a polysulfone, a polyurethane, a polyphenylene oxide, a polyimide, a polyamide, a polyester, a polyether, a polyketone, a polyepoxide, a styrene-acrylonitrile copolymer, a polymethacrylate, a poly(methyl methacrylate), and combinations thereof. In this context, “combination” means mixtures and copolymers thereof. The mixtures and copolymers can include one or more members of the group and/or other monomers/polymers.

For embodiments in which the second polymer has a Tg lower than at least one Tg of the polyurethane, the second polymer is preferably selected from the group consisting of poly(ether urethane), poly(ester urethane), polyester, polyether, polyamides, aliphatic polycarbonate, poly(vinyl ester), poly(vinyl ether), polyacrylate, and poly(methyl acrylate), and combinations thereof. Preferably, the second polymer is not a hydrophobic cellulose ester. Preferred embodiments of the present invention (e.g., those that include a hydrophobic active agent) include a polycarbonate as the second polymer. Suitable polycarbonates are commercially available from Bayer under the trade designation MAKROLON.

If the second polymer is a polyurethane, it is different than the polyurethane discussed above (i.e., the first polymer of the miscible polymer blend). It can be selected from one of the polyurethanes discussed above. Preferably, the second polymer is a polyurethane having a Shore durometer hardness that is higher than that of the first polyurethane. More preferably, the second polymer is a polyurethane having a Shore durometer hardness of about 80D to about 90D. Alternatively, the second polymer can be a polyurethane having a Shore durometer hardness that is lower than that of the first polyurethane. For such embodiments, the second polymer is preferably a polyurethane having a Shore durometer hardness of about 20A to about 80A.

If the active agent is hydrophilic and of low molecular weight (no greater than 1200 g/mol), it is generally undesirable to include a hydrophilic polymer in the system. Although, it can be done, for example, if the system is a reservoir system. In this case, the hydrophilic polymer is in a base coat with a hydrophilic active agent incorporated therein, and with a miscible blend of hydrophobic polymers forming a cap coat, as prepared in Example 5 (although the goal of Example 5 was to prepare a reservoir system, this may not have been achieved due to the method of preparation). The hydrophobic polymers control the delivery of the log molecular weight hydrophilic active agent.

Suitable hydrophilic polymers can be naturally occurring or synthetic. They can include, polypeptides (e.g., proteins, oligopeptides) and polynucleotides (e.g., oligonucleotides, DNA, RNA, and analogs thereof). Examples of suitable hydrophilic polymers include, but are not limited to, polyurethanes, polyvinyl alcohols, poly(alkylene ether)s such as polypropylene oxide, polyethylene oxide, and polytetramethyl oxide, polyvinyl pyridines, polyvinyl pyrrolidones, polyacrylonitriles (at least partially hydrolyzed), polyacrylamides, polyvinyl pyrrolidone/polyvinyl acetate copolymers, sulfonated polystyrenes, polyvinyl pyrrolidone/polystyrene copolymers, polysaccharides such as dextran and mucopolysaccharides, xanthan, hydrophilic cellulose derivatives such as hydroxypropyl cellulose and methyl cellulose, hyaluronic acid, hydrophilic polyacrylates and methacrylates such as polyacrylic acid, polymethacrylic acid, and polyhydroxyethyl methacrylate, DNA and RNA or analogs thereof, heparin, chitosan, polyethylene imine, polyacrylamide, as well as other nitrogen-containing polymers (e.g., amine-containing polymers), and combinations thereof. In this context, “combination” means mixtures and copolymers thereof. The mixtures and copolymers can include one or more members of the group and/or other monomers/polymers.

For certain other embodiments that include a hydrophilic active agent, the hydrophilic polymer is preferably a hydrophilic polyurethane. A preferred hydrophilic polyurethane includes soft segments having therein polyethylene oxide units. Examples of suitable hydrophilic polyurethanes are poly(ether urethanes) available from Thermedics, Inc. (Woburn, Mass.), under the tradename TECOPHILIC.

Preferably, higher molecular weights of polymers are desirable for better mechanical properties; however, the molecular weights should not be so high such that the polymer is not soluble in a processing solvent for preferred solvent-coating techniques or not miscible with the other polymer(s) in the blend. A preferred second polymer has a number average molecular weight of at least about 10,000 g/mol, and more preferably at least about 50,000 g/mol. A preferred second polymer has a number average molecular weight of no greater than about 1,000,000 g/mol, and more preferably no greater than about 500,000 g/mol.

For certain embodiments, preferably, the second polymer has at least one Tg equal to or higher than all Tg's of the polyurethane (first polymer). Preferably, the polyurethane (first polymer) has a hard phase Tg of about 10° C. to about 80° C. (more preferably, about 20° C. to about 60° C.), and the preferred second polymer has at least one Tg (which is of a hard phase if it is a polyurethane) of about 50° C. to about 200° C. (more preferably, about 80° C. to about 150° C.).

Preferred embodiments of the present invention that include a hydrophobic active agent in a matrix system include a combination of a poly(carbonate urethane), which has a Tg of 20-40° C., and a higher durometer poly(carbonate urethane), which has a Tg of 70-90° C. Another preferred combination includes a poly(carbonate urethane), which has a Tg of 10-80° C., and polycarbonate, which has a Tg of 140° C. A third preferred combination includes a poly(ether urethane), which has a Tg of about 22° C., and a phenoxy resin, which has a Tg of 77° C. By combining such high and low Tg polymers, the active agent delivery system can be tuned for the desired dissolution time of the active agent.

Preferred embodiments of the present invention that include a hydrophilic active agent include a combination of a poly(ether urethane), which has a Tg of about 22° C., and a second poly(ether urethane), which has a Tg of 77° C., as a blend that forms a cap coat in a reservoir system. In this embodiment, both polyurethanes are hydrophobic, and both polyurethanes have a solubility parameter greater than 21 J^{1/2}/cm^{3/2}. Thus, they can be used with an active agent that has a similarly matched solubility parameter, even if the active agent is hydrophilic.

Preferably, the difference between the solubility parameter of the active agent and at least one solubility parameter of at least one polymer of the miscible polymer blend is no greater than about 10 J^{1/2}/cm^{3/2} (preferably, no greater than about 5 J^{1/2}/cm^{3/2}, and more preferably, no greater than about 3 J^{1/2}/cm^{3/2}). More preferably, at least one of the following relationships is true: the difference between the solubility parameter of the active agent and the solubility parameter of the polyurethane hard segment is no greater than about 10 J^{1/2}/cm^{3/2} (preferably, no greater than about 5 J^{1/2}/cm^{3/2}, and more preferably, no greater than about 3 J^{1/2}/cm^{3/2}); the difference between the solubility parameter of the active agent and the solubility parameter of the polyurethane soft segment is no greater than about 10 J^{1/2}/cm^{3/2} (preferably, no greater than about 5 J^{1/2}/cm^{3/2}, and more preferably, no greater than about 3 J^{1/2}/cm^{3/2}); and the difference between the solubility parameter of the active agent and at least one solubility parameter of the second polymer (which, if the second polymer is a segmented polymer, is the solubility parameter of the hard and/or soft segment, for example) is no greater than about 10 J^{1/2}/cm^{3/2} (preferably, no greater than about 5 J^{1/2}/cm^{3/2}, and more preferably, no greater than about 3 J^{1/2}/cm^{3/2}). Most preferably, the solubility parameter of the active agent is within about 10 J^{1/2}/cm^{3/2} (preferably, within about 5 J^{1/2}/cm^{3/2}, and more preferably, within about 3 J^{1/2}/cm^{3/2}) of at least one solubility parameter of each polymer of the blend.

Preferably, the difference between at least one solubility parameter of each of at least two polymers of the miscible polymer blend is no greater than about 5 J^{1/2}/cm^{3/2} (preferably, no greater than about 3 J^{1/2}/cm^{3/2}). More preferably, at least one of the following relationships is true: the difference between the solubility parameter of the polyurethane hard segment and at least one solubility parameter of the second polymer (which, if the second polymer is a segmented polymer, is the solubility parameter of the hard and/or soft segment, for example) is no greater than about 5 J^{1/2}/cm^{3/2} (preferably, no greater than about 3 J^{1/2}/cm^{3/2}); and the difference between the solubility parameter of the polyurethane soft segment and at least one solubility parameter of the second polymer (which, if the second polymer is a segmented polymer, is the solubility parameter of the hard and/or soft segment, for example) is no greater than about 5 J^{1/2}/cm^{3/2} (preferably, no greater than about 3 J^{1/2}/cm^{3/2}). Most preferably, if two segmented polymers are used, the difference between the solubility parameters of the hard segments is no greater than about 5 J^{1/2}/cm^{3/2} (preferably, no greater than about 3 J^{1/2}/cm^{3/2}), and the difference between the solubility parameters of the soft segments is no greater than about 5 J^{1/2}/cm^{3/2} (preferably, no greater than about 3 J^{1/2}/cm^{3/2}).

A preferred combination for delivery of a hydrophobic active agent includes a poly(carbonate urethane) and a polycarbonate, which have solubility parameters of 23 J^{1/2}/cm^{3/2} (poly(carbonate urethane hard segment) and 23 J^{1/2}/cm^{3/2}, respectively. Another preferred combination for delivery of a hydrophobic active agent includes a poly(ether urethane) and a linear Bis-phenol A epoxide, which have solubility parameters of 23 J^{1/2}/cm^{3/2} (poly(ether urethane hard segment) and 23 J^{1/2}/cm^{3/2}, respectively. Such values were obtained as described below in Table 1. These blends can be used with active agents such as dexamethasone, which has a solubility parameter of 27 J^{1/2}/cm^{3/2}, based on Hoftyzer and van Kevelen's method and Hoy's method (See Note 1 of Table 1) and 21.1 J^{1/2}/cm^{3/2}, based on the molecular dynamics simulation (See Note 2 of Table 1), and rosiglitazone maleate, which has a solubility parameter of 23 J^{1/2}/cm^{3/2}.

A preferred combination for delivery of a hydrophilic active agent includes a poly(ether urethane) and another poly(ether urethane), which have solubility parameters of 23 J^{1/2}/cm^{3/2} (1^{st} poly(ether urethane hard segment) and 23 J^{1/2}/cm^{3/2} (2^{nd} poly(ether urethane hard segment), respectively. Such values were obtained as described below in Table 2. These blends can be used with active agents such as coumarin, which has a solubility parameter of 27 J^{1/2}/cm^{3/2}, based on the molecular dynamics simulation (See Note 2 of Table 2), even though the polymers are hydrophobic and the active agent is hydrophilic.

TABLE 1
Systems for Hydrophobic Active Agent

| | Solubility | | | | |
|---|---|---|---|---|---|
| | parameter | | | | |
| Polymers | (J^{1/2}/cm^{3/2}) | Source | Notes | Tg (° C.) | Source |
| Poly(bisphenyl A carbonate) | 23 | 1 | H-vK, carbonate OCOO = | 140 | 1 |
| | | | COO + O; Hoy OCOO = O + | | |
| | | | COO. Fedors volume 174 | | |
| | | | cm³/mol | | |
| Poly(ether urethane) | 23 | 1 | Methyl diisocyanate (MDI) | | |
| (PELLETHANE 75D) hard | | | and butydiol (BDO) were | | |
| segment | | | used. H-vK, HNCOO = NH + | | |
| | | | COO. Fedors volume 230.3 | | |
| | | | cm³/mol. | | |
| Poly(carbonate urethane) | 23 | 1 | It was assumed to have the | | |
| (BIONATE 75D) hard | | | same structure as | | |
| segment | | | PELLETHANE 75D has. | | |
| | | | Therefore, the calculation | | |
| | | | was the same as that for the | | |
| | | | above PELLETHANE 75D. | | |
| Phenoxy | 23 | 1 | Fedors volume 201 cm³/mol | 95 | Vendor |
| Dexamethasone | 27 | 1 | All rings were treated as | | |
| | | | aliphatic. Hydroxyl groups | | |
| | | | were not involved in | | |
| | | | hydrogen bonding. Fedors | | |
| | | | volume 205 cm³/mol | | |
| | 21 | 2 | | | |
| Rosiglitazone maleate | 24 | 1 | H-vK, C5NH5 = C6H5*5/6 + | | |
| | | | tertiary N, CONHCO as 2CO + | | |
| | | | NH; Hoy, aromatic tertiary | | |
| | | | N treated as aliphatic tertiary | | |
| | | | N, CONHCO as CONH + CO. | | |
| | | | Fedors volume 306 cm³/mol. | | |

Source for Solubility Parameters:

1. Average of the calculated values based on Hoftyzer and van Kevelen's (H-vK) method (where the volumes of the chemicals were calculated based on Fedors' method) and Hoy's method. See Chapter 7, D. W. van Krevelen, Properties of Polymers, 3rd ed., Elsevier, 1990, for details of all the calculations, where Table 7.8 was for Hoftyzer and van Kevelen's method, Table 7.3 for Fedors' method, and Table 7.9 and 7.10 for Hoy's method.

2. Values based on the molecular dynamics simulation with Accelrys Materials Studio, Accelrys Inc., San Diego, Calif. Simulation began with building molecular models with Atomistic Tool. The atoms of the drug were assigned groupsed based their charges. After minimizing the energy of the molecule, amorphous cells that contained a number of molecules were built (total number of atoms of each cell was no more than 9500). Energy minimizations were conducted to eliminate any strain that occurred during the amorphous cell building. Dynamics simulations were consequently conducted for a simulated time of about 200 ps. The cohesive energy density and solubility parameter were calculated based on about 5 configurations the final stages of the simulation. COMPASS force field was used.

Source of Tg's (the Reported Value is the Average if there are Two Values Listed in the Sources):

1. Table 6.6, M. J. He, W. X. Chen, and X. X. Dong, Polymer Physics, revised version, FuDan University Press, ShangHai, China, 2000. Data were the average if there were two values listed in the sources.

TABLE 2
System for Hydrophilic Active Agent

| | Solubility | | | | |
|---|---|---|---|---|---|
| | parameter | | | Tg (° C.) | |
| Polymers | (J^{1/2}/cm^{3/2}) | Source | Notes | DSC | Source |
| Poly(ether urethane) | 23 | 1 | Methyl diisocyanate (MDI) | 22 | See |
| (PELLETHANE 75D) hard | | | and butydiol (BDO) were | | Example 5 |
| segment | | | used. H-vK, HNCOO = NH + | | |
| | | | COO. Fedors volume 230.3 | | |
| | | | cm³/mol. | | |
| Poly(ether urethane) | 23 | 1 | Methyl diisocyanate (MDI), | 72 | |
| (TECOPLAST TP-470) hard | | | hexanediol (CDO), and | | |
| segment | | | cyclohexanediol (CHDO) | | |
| | | | were used. H-vK, HNCOO = | | |
| | | | NH + COO. | | |
| Coumarin | 24 | 2 | Molecular Dynamics | | |
| | | | Simulation with Accelrys | | |
| | | | Molecular Studio | | |

Source for Solubility Parameters:

1. Average of the calculated values based on Hoftyzer and van Kevelen's (H-vK) method (where the volumes of the chemicals were calculated based on Fedors' method) and Hoy's method. See Chapter 7, D. W. van Krevelen, Properties of Polymers, 3rd ed., Elsevier, 1990, for details of all the calculations, where Table 7.8 was for Hoftyzer and van Kevelen's method, Table 7.3 for Fedors' method, and Table 7.9 and 7.10 for Hoy's method.

2. Values based on the molecular dynamics simulation with Accelrys Materials Studio, Accelrys Inc., San Diego, Calif. Simulation began with building molecular models with Atomistic Tool. The atoms of the drug were grouped based their charges. After minimizing the energy of the molecule, amorphous cells that contained a number of molecules were built (total number of atoms of each cell was no more than 9500). Energy minimizations were conducted to eliminate any strain that occurred during the amorphous cell building. Dynamics simulations were consequently conducted for a simulated time of about 200 ps. The cohesive energy density and solubility parameter were calculated based on about 5 configurations the final stages of the simulation. COMPASS force field was used.

The polymers in the miscible polymer blends can be crosslinked or not. Similarly, the blended polymers can be crosslinked or not. Such crosslinking can be carried out by one of skill in the art after blending using standard techniques.

In the active agent systems of the present invention, the active agent passes through a miscible polymer blend having a “critical” dimension. This critical dimension is along the net diffusion path of the active agent and is preferably no greater than about 1000 micrometers (i.e., microns), although for shaped objects it can be up to about 10,000 microns.

For embodiments in which the miscible polymer blends form coatings or free-standing films (both generically referred to herein as “films”), the critical dimension is the thickness of the film and is preferably no greater than about 1000 microns, more preferably no greater than about 500 microns, and most preferably no greater than about 100 microns. A film can be as thin as desired (e.g., 1 nanometer), but are preferably no thinner than about 10 nanometers, more preferably no thinner than about 100 nanometers. Generally, the minimum film thickness is determined by the volume that is needed to hold the required dose of active agent and is typically only limited by the process used to form the materials. For all embodiments herein, the thickness of the film does not have to be constant or uniform. Furthermore, the thickness of the film can be used to tune the duration of time over which the active agent is released.

For embodiments in which the miscible polymer blends form shaped objects (e.g., microspheres, beads, rods, fibers, or other shaped objects), the critical dimension of the object (e.g., the diameter of a microsphere or rod) is preferably no greater than about 10,000 microns, more preferably no greater than about 1000 microns, even more preferably no greater than about 500 microns, and most preferably no greater than about 100 microns. The objects can be as small as desired (e.g., 10 nanometers for the critical dimension). Preferably, the critical dimension is no less than about 100 microns, and more preferably no less than about 500 nanometers.

In one embodiment, the present invention provides a medical device characterized by a substrate surface overlayed with a polymeric top coat layer that includes a miscible polymer blend, preferably with a polymeric undercoat (primer) layer. When the device is in use, the miscible polymer blend is in contact with a bodily fluid, organ, or tissue of a subject.

The invention is not limited by the nature of the medical device; rather, any medical device can include the polymeric coating layer that includes the miscible polymer blend. Thus, as used herein, the term “medical device” refers generally to any device that has surfaces that can, in the ordinary course of their use and operation, contact bodily tissue, organs or fluids such as blood. Examples of medical devices include, without limitation, stents, stent grafts, anastomotic connectors, leads, needles, guide wires, catheters, sensors, surgical instruments, angioplasty balloons, wound drains, shunts, tubing, urethral inserts, pellets, implants, pumps, vascular grafts, valves, pacemakers, and the like. A medical device can be an extracorporeal device, such as a device used during surgery, which includes, for example, a blood oxygenator, blood pump, blood sensor, or tubing used to carry blood, and the like, which contact blood which is then returned to the subject. A medical device can likewise be an implantable device such as a vascular graft, stent, stent graft, anastomotic connector, electrical stimulation lead, heart valve, orthopedic device, catheter, shunt, sensor, replacement device for nucleus pulposus, cochlear or middle ear implant, intraocular lens, and the like. Implantable devices include transcutaneous devices such as drug injection ports and the like.

In general, preferred materials used to fabricate the medical device of the invention are biomaterials. A “biomaterial” is a material that is intended for implantation in the human body and/or contact with bodily fluids, tissues, organs and the like, and that has the physical properties such as strength, elasticity, permeability and flexibility required to function for the intended purpose. For implantable devices in particular, the materials used are preferably biocompatible materials, i.e., materials that are not overly toxic to cells or tissue and do not cause undue harm to the body.

The invention is not limited by the nature of the substrate surface for embodiments in which the miscible polymer blends form polymeric coatings. For example, the substrate surface can be composed of ceramic, glass, metal, polymer, or any combination thereof. In embodiments having a metal substrate surface, the metal is typically iron, nickel, gold, cobalt, copper, chrome, molybdenum, titanium, tantalum, aluminum, silver, platinum, carbon, and alloys thereof. A preferred metal is stainless steel, a nickel titanium alloy, such as NITINOL, or a cobalt chrome alloy, such as NP35N.

A polymeric coating that includes a miscible polymer blend can adhere to a substrate surface by either covalent or non-covalent interactions. Non-covalent interactions include ionic interactions, hydrogen bonding, dipole interactions, hydrophobic interactions and van der Waals interactions, for example.

Preferably, the substrate surface is not activated or functionalized prior to application of the miscible polymer blend coating, although in some embodiments pretreatment of the substrate surface may be desirable to promote adhesion. For example, a polymeric undercoat layer (i.e., primer) can be used to enhance adhesion of the polymeric coating to the substrate surface. Suitable polymeric undercoat layers are disclosed in Applicants' copending U.S. Provisional Application Serial No. 60/403,479, filed on Aug. 13, 2002, and U.S. patent application Ser. No. ______, filed on even date herewith, both entitled MEDICAL DEVICE EXHIBITING IMPROVED ADHESION BETWEEN POLYMERIC COATING AND SUBSTRATE. A particularly preferred undercoat layer disclosed therein consists essentially of a polyurethane. Such a preferred undercoat layer includes a polymer blend that contains polymers other than polyurethane but only in amounts so small that they do not appreciably affect the durometer, durability, adhesive properties, structural integrity and elasticity of the undercoat layer compared to an undercoat layer that is exclusively polyurethane.

When a stent or other vascular prosthesis is implanted into a subject, restenosis is often observed during the period beginning shortly after injury to about four to six months later. Thus, for embodiments of the invention that include stents, the generalized dissolution rates contemplated are such that the active agent should ideally start to be released immediately after the prosthesis is secured to the lumen wall to lessen cell proliferation. The active agent should then continue to dissolute for up to about four to six months in total.

The invention is not limited by the process used to apply the polymer blends to a substrate surface to form a coating. Examples of suitable coating processes include solution processes, powder coating, melt extrusion, or vapor deposition.

A preferred method is solution coating. For solution coating processes, examples of solution processes include spray coating, dip coating, and spin coating. Typical solvents for use in a solution process include tetrahydrofuran (THF), methanol, ethanol, ethylacetate, dimethylformamide (DMF), dimethyacetamide (DMA), dimethylsulfoxide (DMSO), dioxane, N-methyl pyrollidone, chloroform, hexane, heptane, cyclohexane, toluene, formic acid, acetic acid, and/or dichloromethane. Single coats or multiple thin coats can be applied.

Similarly, the invention is not limited by the process used to form the miscible polymer blends into shaped objects. Such methods would depend on the type of shaped object. Examples of suitable processes include extrusion, molding, micromachining, emulsion polymerization methods, electrospray methods, etc.

For preferred embodiments in which the active agent delivery system includes one or more coating layers applied to a substrate surface, a preferred embodiment includes the use of a primer, which is preferably applied using a “reflow method,” which is described in Applicants' copending U.S. Provisional Application Serial No. 60/403,479, filed on Aug. 13, 2002, and U.S. patent application Ser. No. ______, filed on even date herewith, both entitled MEDICAL DEVICE EXHIBITING IMPROVED ADHESION BETWEEN POLYMERIC COATING AND SUBSTRATE.

Preferably, in this “reflow method,” the device fabrication process involves first applying an undercoat polymer to a substrate surface to form the polymeric undercoat layer, followed by treating the polymeric undercoat layer to reflow the undercoat polymer, followed by applying a miscible polymer blend, preferably with an active agent incorporated therein, to the reformed undercoat layer to form a polymeric top coat layer. Reflow of the undercoat polymer can be accomplished in any convenient manner, e.g., thermal treatment, infrared treatment, ultraviolet treatment, microwave treatment, RF treatment, mechanical compression, or solvent treatment. To reflow the undercoat polymer, the undercoat layer is heated to a temperature that is at least as high as the “melt flow temperature” of the undercoat polymer, and for a time sufficient to reflow the polymer. The temperature at which the polymer enters the liquid flow state (i.e., the “melt flow temperature”) is the preferred minimum temperature that is used to reflow the polymer according to the invention. Typically 1 to 10 minutes is the time period used to reflow the polymer using a thermal treatment in accordance with the invention. The melt flow temperature for a polymer is typically above the Tg (the melt temperature for a glass) and the Tm (the melt temperature of a crystal) of the polymer.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

Poly(carbonate urethane)/Poly(bis-phenol A carbonate) Blends with Dexamethasone (Hydrophobic Active Agent)

Blend Preparation and Miscibility Testing

Poly(carbonate urethane) 75D (PCU 75D) was purchased from Polymer Technology Group, Inc., Berkeley, Calif. It is a copolymer of hydroxyl terminated polycarbonate, aromatic diisocyanate, and low molecular weight glycol. Poly(bis-phenol A carbonate) (PC), having a melt index (300° C./1.2 kg, ASTM D 1238) of 7 grams/10 minutes, was purchased from Sigma-Aldrich Co., Milwaukee, Wis. Prior to blending, the two polymers were dried at 60° C. to 70° C. at reduced pressure overnight. The two dried polymers were dry-mixed at various ratios, followed by melt blending at about 200-225° C. with a batch mixer (ThermoHaake, Karlsruhe, BW, Germany) equipped with two roller blades. The blending was conducted at 50 revolutions per minute (rpm). When the torque leveled off (within 2 to 3 minutes), the rpm was increased to 100. After the torque leveled off again (within 2 to 3 minutes), the rpm was set back to 50 rpm. Blending was continued for 1 more minute. After mixing was complete, the samples were collected and cooled to room temperature in air. In order to prevent oxidation during blending, 0.1-0.2 wt-% of IRGANOX 1010 antioxidant (Ciba Specialties Chemical Co., Terrytown, N.Y.) was added into the blends before melt mixing.

The miscibility between PCU 75D and PC was tested by measuring the thermal transition temperatures of the blends from their mechanical properties. Film samples were prepared by pressing the blend samples between two hot plates at about 230° C. for about 5 minutes. Typically, the films were about 0.1 millimeter (mm) to 0.5 mm thick, 5 mm to 7 mm wide, and 2 centimeters (cm) to 3 cm long. These films were mounted in a film/fiber fixture of a Rheometric Solids Analyzer III (RSAIII) (Rheometric Scientific, Inc., Piscataway, N.J.). The initial gap was set to about 5 mm. Tests were done in dynamic mode at a frequency of 1 Hz. The mechanical properties were recorded during heating the sample at a rate of 5° C./minute from −80° C. to 200° C. The commanded strain was set to 0.1% from −80° C. to 0° C., 0.5% from 0° C. to 150° C., and 1% from 150° C. to 200° C.

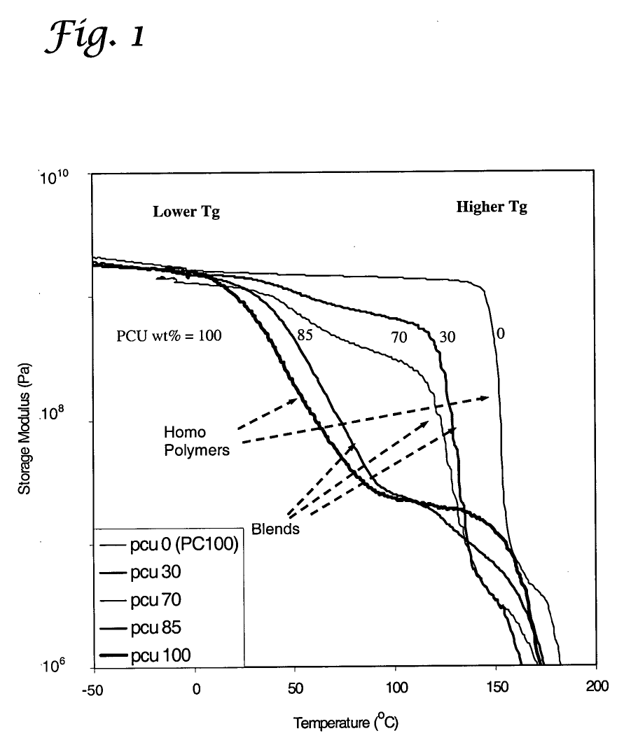

FIG. 1 shows the storage modulus versus temperature. The modulus of pure PC started to drop at about 140° C. Therefore, the Tg of PC was about 140° C. Pure PCU had a similar transition that started at about 10° C. until about 80° C. For the blends containing both PCU and PC, there were two glass transitions. As the content of PC increased, both of the Tg's increased and became closer together. This suggested that the PCU and PC were miscible.

Sample Preparation with Dexamethasone

Dissolution samples were prepared by solvent blending. Before dissolving PCU 75D poly(carbonate urethane) in THF, it was dried overnight at 70° C. under reduced pressure, then melted and pressed between two hot plates at 230° C. for 5-10 minutes. Then the films were cooled and placed in anhydrous tetrahydrofuran (THF) at about 60° C. The mixture was stirred with a magnetic bar until the polymer was dissolved. A small amount of gel was occasionally detected in solution, which was removed by filtering the solution with a 0.45-micron (μm) filter. The concentration of PCU was 1.16 wt-%. The PC was first dissolved in chloroform at room temperature to make a 5 wt-% solution. Then the solution was diluted with anhydrous THF to 1 wt-%. A 1 wt-% solution of dexamethasone (Sigma-Aldrich) in anhydrous THF was also made at room temperature. Then the three solutions were mixed at varying ratios to make different samples with the compositions shown in Table 3.

TABLE 3

| | | | | | | | |
|---|---|---|---|---|---|---|---|
| PCU/PC | 100/0 | 90/10 | 80/20 | 70/30 | 50/50 | 30/70 | 0/100 |
| (weight ratio) | | | | | | | |
| Dexamethasone | 9.7 | 8.3 | 9.7 | 1.0 | 8.9 | 9.2 | 12.0 |
| (wt-%) based on | | | | | | | |
| total solids | | | | | | | |

Dissolution samples were prepared with stainless steel (316L) shims that were cleaned by rinsing with THF. The cleaned shims were coated with a solution of 1 wt-% poly(ether urethane) (PELLETHANE 75D, Dow Chemical Co., Midland, Mich.) dissolved in THF. Before dissolving PELLETHANE 75D poly(ether urethane) in THF, it was dried overnight at 70° C. under reduced pressure, then melted and pressed between two hot plates at 230° C. for 5-10 minutes. Then the films were cooled and dissolved in anhydrous tetrahydrofuran (THF) at about 25° C. by stirring with a magnetic bar overnight.

The coated shims were allowed to dry overnight under nitrogen. Subsequently, they were thermally treated at 215-220° C. for 5-10 minutes. This pre-treatment led to formation of a primer on the surface of the shims that promoted their adhesion with polymer/active agent layers. The primer-treated shims were coated with the solutions listed above and dried overnight under nitrogen. The shims were weighed after each step. Based on the weight difference, the total amount of polymer/active agent coating was determined as was the thickness of the coating. A typical coating thickness was about 10 microns.

Dissolution of Dexamethasone

Dissolution of dexamethasone from PCU 75D/PC polymer matrix was conducted by placing the coated shims in glass vials that contained phosphate buffered saline solution (PBS, potassium phosphate monobasic (NF tested), 0.144 grams per liter (g/L), sodium chloride (USP tested), 9 g/L, and sodium phosphate dibasic (USP tested) 0.795 g/L, pH=7.0 to 7.2 at 37° C., purchased from HyClone, Logan, Utah). Each shim had about 2 milligrams (mg) of coating (about 0.2 mg of dexamethasone) and each vial contained 3 milliliters (mL) of PBS. The vials were stored in an incubator-shaker at 37° C. and agitated at about 50 revolutions per minute (rpm). The PBS was collected from the vials and replaced with fresh PBS. The concentration of dexamethasone was measured with a UV-Vis spectrophotometer (HP 4152A) that was calibrated with a series of dexamethasone solutions with known concentrations.

Dissolution Data Analysis

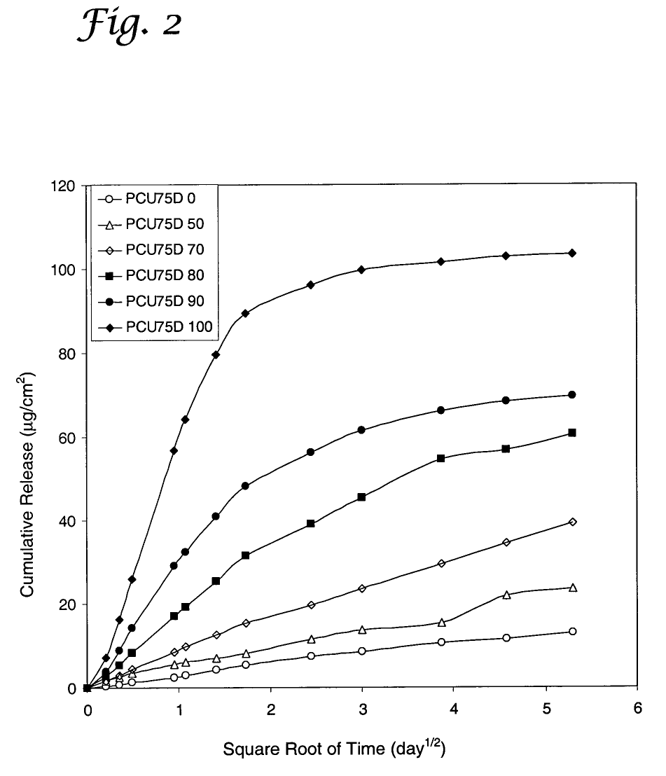

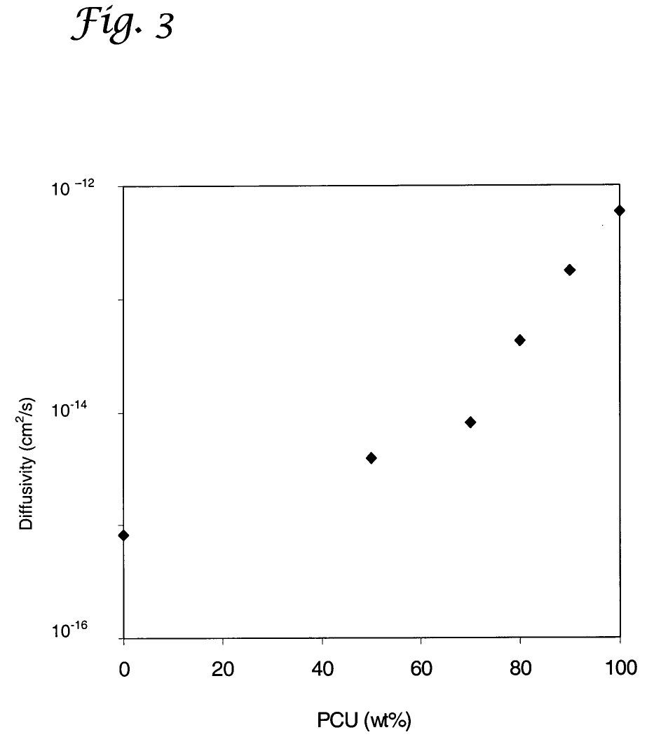

FIG. 2 shows the cumulative release of dexamethasone increased with an increasing amount of PCU in the blend. These release curves clearly show that the release rate of dexamethasone could be adjusted by varying the content of PCU in the blends. Based on the curves, the diffusion coefficients of dexamethasone from these blends were calculated using the following equation and plotted as a function of blend composition in FIG. 3.

$$D = {( \frac{{M}_{t}}{4 {M}_{∞}} )}^{2} \cdot \frac{π {x}^{2}}{t}$$

wherein D=diffusion coefficient; Mₜ=cumulative release; M∞=total loading of active agent; x=the critical dimension (e.g., thickness of the film); and t=the dissolution time.

FIG. 3 shows the log of the diffusion coefficient was almost a linear function of the blend composition, which demonstrated that the active agent release rate can be tuned by using miscible polymer blends. Additionally, the data presented in FIG. 2 shows no burst, which indicates that the release of the active agent was predominantly under permeation control.

### Example 2

Poly(ether urethane)/Phenoxy Blends with Dexamethasone (Hydrophobic Active Agent)

Poly (ether urethane) (PELLETHANE 75D) and dexamethasone were the same as that used in Example 1. Phenoxy resin (PX), a linear poly(bis-phenol A epoxide), was obtained from the Phenoxy Specialties Corp., Rockhill, Calif.). The grade used in the present example was PKHJ with a number average molecular weight of about 10-16 kilograms per mole (Kg/mol) and a Tg of 95° C. This material was expected to slow down the release rate of dexamethasone as the PC did in Example 1.

PELLETHANE 75D and dexamethasone were dissolved in THF as described in Example 1 (all the following procedures were the same as those used in Example 1 if not specified). PX was dissolved in anhydrous THF at room temperature with 1 wt-% of polymer in the solution. These three solutions were mixed at various ratios and coated onto stainless steel shims that were primer-treated in the same procedure as described in Example 1. After the coating dried, dissolution and UV-Vis analysis were conducted.

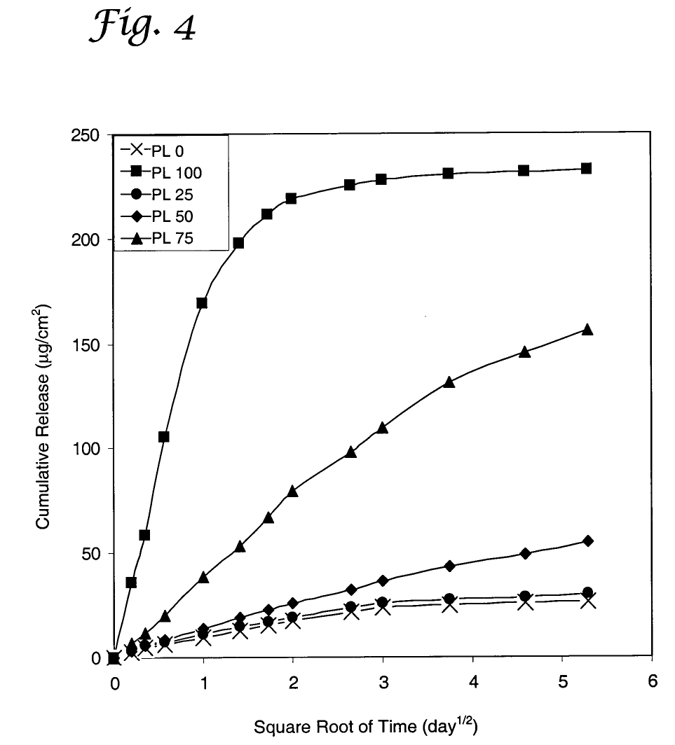

Cumulative release of dexamethasone from the PELLETHANE 75D/PX blend matrix was plotted in FIG. 4. The release rate of dexamethasone increased with an increasing amount of PELLETHANE 75D in the blend. These release curves clearly show that by varying the contents of PELLETHANE 75D and PX, the release rate of dexamethasone was tuned. Additionally, the data presented in FIG. 4 shows no burst, which indicates that the release of the active agent was predominantly under permeation control.

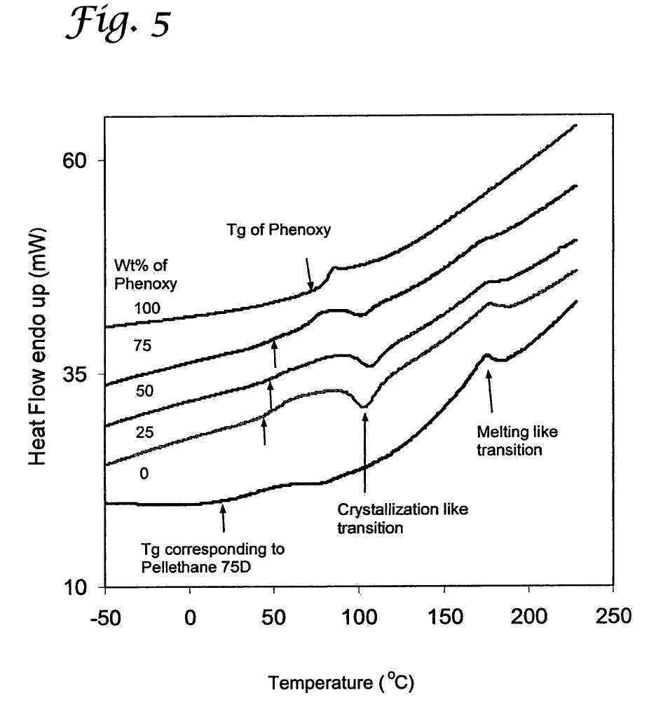

Miscibility between PELLETHANE 75D and PX was tested by measuring the Tg transitions of the PELLETHANE 75D/PX blends with a PYRIS 1 differential scanning calorimeter (DSC), PerkinElmer Company, Wellesley, Mass. Solutions of about 5 wt-% PELLETHANE 75D and PX in THF were made separately using the same procedure as described above. The blend samples, each about 10 mg, were loaded into the DSC and were scanned from −100° C. to 230° C. at 40° C./minute. Each sample was scanned twice. The second scan had less noise and was used. PYRIS software version 5.0 was used to determine the onset of Tg transitions. As shown in FIG. 5, the pure PELLETHANE 75D had a glass transition at about 22° C. and a melt-like transition at about 173° C. This Tg was considered to be associated with the hard domain of the resin. The Tg of the soft domain of poly(ether urethane), if it can be detected, is usually below 0° C. The pure PX had a Tg transition at a higher temperature (77° C.). When PELLETHANE 75D and PX were blended, there were two changes. First, the Tg transitions of the pure PELLETHANE 75D and PX could no longer be clearly identified from the blend samples. There was a broader Tg transition range with a higher onset temperature compared to the Tg of the pure PELLETHANE 75D. This suggests that PELLETHANE 75D and PX are at least partially miscible (as defined herein). Second, there was a new transition representative of a crystalline component immediately after the Tg transition in all three blends. This suggests that PX caused a faster crystallization transition in PELLETHANE 75D, indicating the presence of interactions between PX and PELLETHANE 75D hard domains. This further supports the miscibility between the two materials.

### Example 3

Poly(carbonate urethane) 75D/ Poly(carbonate urethane) 55D Blends with Dexamethasone (Hydrophobic Active Agent)

Poly(carbonate urethane) 75D (PCU 75D) and dexamethasone solutions were the same as that used in Example 1. PCU 55D is the trade designation for another member of the poly(carbonate urethane) family made by the Polymer Technology Group but softer than the PCU 75D polymer. It was dissolved in anhydrous THF in a similar procedure as that described in Example 1 for PCU 75D except the dissolution occurred at room temperature rather at 60° C. These three solutions were mixed at various ratios, coated onto stainless steel shims, and dried using the same procedures described in Example 1. Dissolution tests were conducted as described in Example 1.

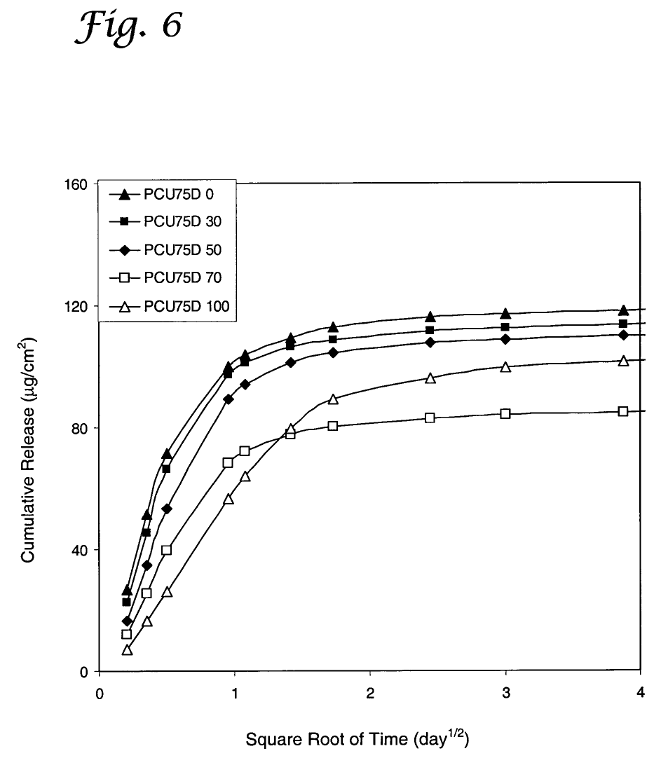

Cumulative release of dexamethasone from the PCU 75D/PCU 55D blends is shown in FIG. 6. The release rate of dexamethasone increased with an increasing amount of PCU 55D in the blend. These release curves clearly show that by blending a softer (i.e., lower durometer) PCU into a harder one, the release rates of active agent could be increased.

It should be pointed out that the crossover between PCU 75D 100 with PCU 75D 70 was due to the thickness difference of the two. The release rates were determined by the initial linear region but not the later flat portions of the curves. Dexamethasone was released faster from PCU 75D 100 than from PCU 75D 70.

### Example 4

Poly(ether urethane)/Phenoxy Resin with Rosiglitazone maleate (Hydrophobic Active Agent)

Rosiglitazone maleate, commercially available from Smithkline Beecham, United Kingdom, was released from PELLETHANE 75D/PX blends as described in Example 2. The blend compositions and all the sample preparation and test procedures were the same as those described in Example 2.

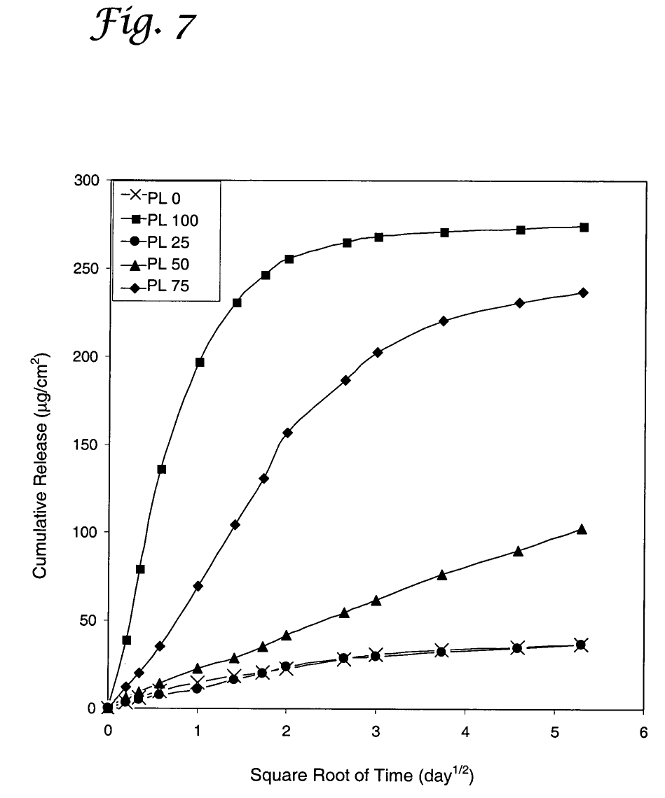

Cumulative release of this active agent was plotted in FIG. 7. The release rate increased with an increasing amount of PELLETHANE 75D in the blend. These release curves clearly show that the release rate of rosiglitazone maleate was tuned by using miscible polymer blends.

### Example 5

Poly(ether urethane) Blends with Coumarin (Hydrophilic Active Agent)

PELLETHANE 75D (PL75D), a poly(ether urethane), was purchased from Dow Chemical Company, Midland, Mich. TECOPLAST (TP) (TP-470) and TECOPHILIC (TL) 60D60, other two poly(ether urethane)s, were purchased from Thermedics, Inc., Woburn, Mass. TP has a Shore Hardness of 82D. Coumarin was purchased from Sigma-Aldrich Co., Milwaukee, Wis. Based on the Merck Index (13 edit., Merck & CO., INC., Whitehouse Station, N.J.), one gram of coumarin dissolves in 400 mL of cold water. Anhydrous tetrahydrofuran (THF), anhydrous methanol, and acetonitrile (HPLC) used in this example were also purchased from Sigma-Aldrich Co., Milwaukee, Wis.

PL75D was dried at 70° C. at reduced pressure overnight. The dried pellets were compressed between two plates that were pre-heated to 230° C. and maintained for about 5 minutes. After the sample was cooled down to room temperature, it was placed in a vial filled with THF and stirred until dissolved (by visual observation). TP and TL were directly dissolved in THF by stirring the mixtures at room temperature. Coumarin was also dissolved in THF. The concentrations of all the solutions were about 1 wt-%. TL solution and coumarin solution were mixed at a weigh ratio of 1:1. This mixture is the base coating solution of a reservoir system. TP solution and PL75D solution were mixed at various weight ratios to make five different mixtures with the weight ratios of TP to PL75D being 100:0, 75:25, 50:50, 25:75, and 0:100. These solutions are referred to herein as cap coating solutions of the reservoir system.

Dissolution samples were prepared with stainless steel (316L) shims (12.1×38.1 mm²) that were cleaned by rinsing with THF. The cleaned shims were coated with the PL75D/THF solution. The coated shims were allowed to dry overnight under nitrogen. Subsequently, they were thermally treated at 215-220° C. for 5-10 minutes. This thermal treatment led to formation of a primer on the surface of the shims that promoted their adhesion with polymer/active agent layers. The thickness of the primer coating was about 1 micrometer (micron). Five primer-treated shims were then coated with the base coating solution and dried overnight. Then, these shims were dip-coated with different cap coating solutions in the following way: the shim was dipped into one of the cap coating solutions for 2 to 3 seconds then was dried in nitrogen gas (for about 1 minute). Such dipping and drying processes were repeated for 8 times for each shim. The whole processes were completed in a nitrogen filled glove box. After completion of the coating, all five shims with different cap coating solutions were further dried in the glove box overnight. The thickness of the cap coating in each shim was about 1.7 to 3.4 microns. All the coatings were clear and transparent.

Dissolution of Coumarin

Dissolution of coumarin from the cap-coated shims was conducted by placing the coated shims in glass vials that contained phosphate buffered saline solution (PBS, potassium phosphate monobasic (NF tested), 0.144 grams per liter (g/L), sodium chloride (USP tested), 9 g/L, and sodium phosphate dibasic (USP tested) 0.795 g/L, pH=7.0 to 7.2 at 37° C., purchased from HyClone, Logan, Utah). Each vial contained 4 milliliters (mL) of PBS. The vials were stored in an incubator-shaker at 37° C. and agitated at about 50 rpm. The PBS was collected from the vials and replaced with fresh PBS at predetermined times. After one week, the dissolution tests were stopped and the remaining coating were dissolved in 4 mL of acetonitrile. The concentration of coumarin in all these solutions was measured with a liquid chromatography (HP 1090) that was equipped with a UV detector. Mobile phase was a mixture of 50 wt-% of sodium acetate water solution (pH=4) with 50 wt-% of acetonitrile (HPLC). The flow rate was 1.0 mL/minute. A Zorbax Eclipse (5 micron) column was used. The UV detection was conducted at a wavelength of 277 nm. The standard curve was obtained with a series of coumarin solutions with known concentrations. These standard coumain solutions were made by dissolving coumarin in methanol to make a concentrated solution (about 1 wt-%) and diluting this concentrated solution with PBS.

Dissolution Data Analysis

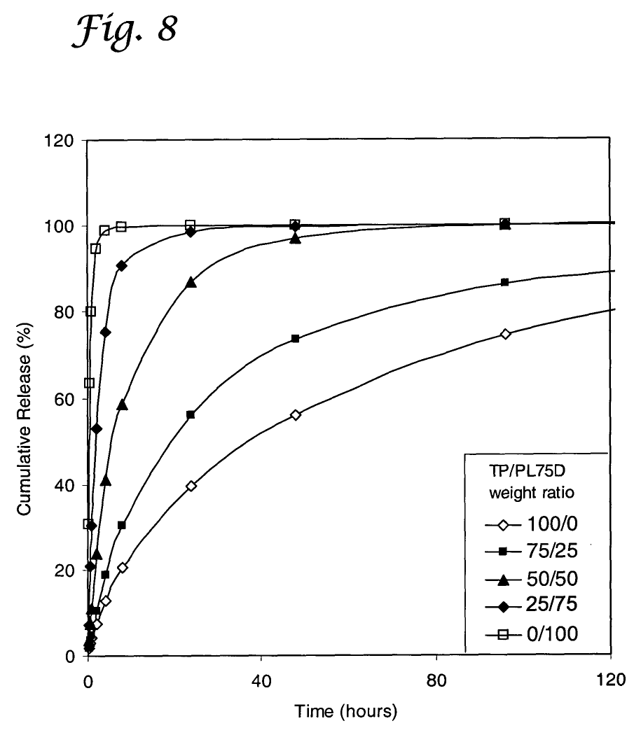

Cumulative percentage release of coumarin versus the PL75D content in the cap-coated shims was plotted in FIG. 8. The total amounts of coumarin in the shims were determined by adding together all the coumarin in dissoluted solutions and that left in the remaining coatings. As was shown in the plot, coumarin was release much faster from a 100% PL75D coated shim than that from 100% TP coated shim. The release rates from the PL75D/TP blends were between that from the two pure polymers. More interestingly, the rate was parallel to the PL75D content in the blends. These results clearly show that the release rate of coumarin could be adjusted by varying the composition of the blends.

Because the PL75D/TP blends were coated as a cap coating on the top of the TL/coumarin layer, we expected there would be time lag in the release curves. However, the result in FIG. 8 did not show this. We speculate that this was because the TL/coumarin was re-dissolved during the dip coating process.

Miscibility Tests

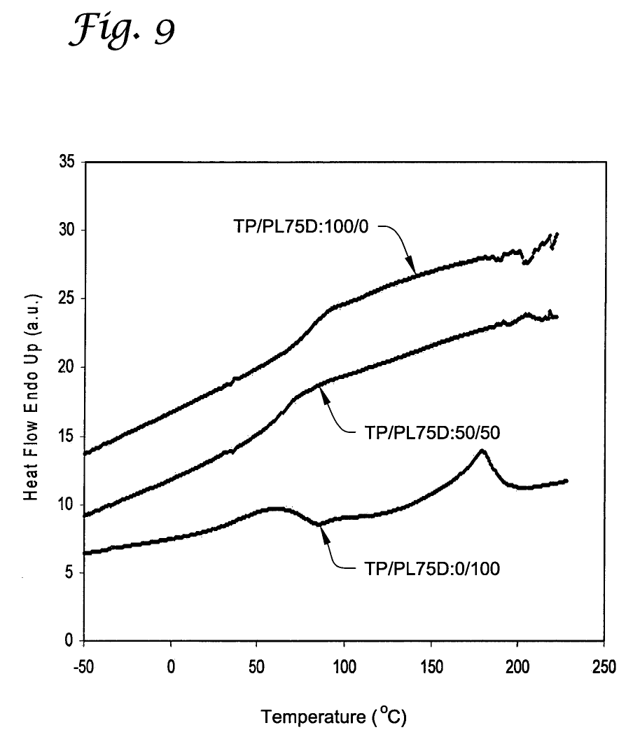

The samples for miscibility tests were made to contain the same TP/PL75D ratios as the dissolution samples had. There was no coumarin in these samples. The samples were scanned with a PYRIS 1 differential scanning calorimeter (DSC) (PerkinElmer Company, Wellesley, Mass.). The scanning was programmed from −100° C. to 220° C. at 40° C./min. The sample size was about 10 milligrams (mg) to 16 mg. As shown in FIG. 9, the pure PL75D had a Tg transition at about 22° C. and a melt-like transition at about 173° C. This Tg was considered to be associated with the hard domain of the resin. The pure TP had a glass transition at about 72° C. When PL75D and TP were blended at a weight ratio of 50/50, there was only one Tg transition that was at about 50° C. This suggested that the PL75D and TP are miscible at this ratio.

The complete disclosures of all patents, patent applications including provisional patent applications, and publications, and electronically available material cited herein are incorporated by reference. The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

What is claimed is:

1. An active agent delivery system comprising an active agent and a miscible polymer blend comprising a polyurethane and a second polymer, wherein the miscible polymer blend controls the delivery of the active agent, wherein the second polymer is not a hydrophobic cellulose ester.

2. The system of claim 1 wherein the second polymer has at least one Tg equal to or higher than all Tg's of the polyurethane.

3. The system of claim 1 wherein the active agent is not heparin.

4. The system of claim 1 wherein the second polymer is selected from the group consisting of a polycarbonate, a polysulfone, a polyurethane, a polyphenylene oxide, a polyimide, a polyamide, a polyester, a polyether, a polyketone, a polyepoxide, a styrene-acrylonitrile copolymer, a polymethacrylate, a poly(methyl methacrylate), and combinations thereof.

5. The system of claim 1 wherein the active agent is incorporated within the miscible polymer blend.

6. The system of claim 5 wherein the active agent is present in the miscible polymer blend in an amount of about 0.1 wt-% to about 80 wt-%, based on the total weight of the miscible polymer blend and the active agent.

7. The system of claim 1 wherein miscible polymer blend initially provides a barrier for permeation of the active agent.

8. The system of claim 7 wherein the active agent is incorporated within an inner matrix.

9. The system of claim 8 wherein the active agent is present in the inner matrix in an amount of about 0.1 wt-% to about 100 wt-%, based on the total weight of the inner matrix including the active agent.

10. The system of claim 1 wherein the polyurethane has a Shore durometer hardness of about 70D to about 80D.

11. The system of claim 10 wherein the second polymer is a polyurethane having a Shore durometer hardness of about 80D to about 90D.

12. The system of claim 10 wherein the second polymer is a polycarbonate.

13. The system of claim 10 wherein the polyurethane is a poly(carbonate urethane) or a poly(ether urethane).

14. The system of claim 13 wherein the active agent is hydrophobic.

15. The system of claim 13 wherein the polyurethane is a poly(ether urethane) and the active agent is hydrophilic.

16. The system of claim 1 wherein:
the active agent has a solubility parameter, the polyurethane has a soft segment solubility parameter and a hard segment solubility parameter, and the second polymer has at least one solubility parameter; and
at least one of the following relationships is true:
the difference between the solubility parameter of the active agent and the solubility parameter of the polyurethane hard segment is no greater than about 10 J^{1/2}/cm^{3/2};
the difference between the solubility parameter of the active agent and the solubility parameter of the polyurethane soft segment is no greater than about 10 J^{1/2}/cm^{3/2}; and
the difference between the solubility parameter of the active agent and at least one solubility parameter of the second polymer is no greater than about 10 J^{1/2}/cm^{3/2}.

17. The system of claim 1 wherein:
the polyurethane has a soft segment solubility parameter and a hard segment solubility parameter, and the second polymer has at least one solubility parameter; and
at least one of the following relationships is true:
the difference between the solubility parameter of the polyurethane hard segment and at least one solubility parameter of the second polymer is no greater than about 5 J^{1/2}/cm^{3/2}; and
the difference between the solubility parameter of the polyurethane soft segment and at least one solubility parameter of the second polymer is no greater than about 5 J^{1/2}/cm^{3/2}.

18. The system of claim 1 wherein the active agent is hydrophobic and has a molecular weight of no greater than about 1200 g/mol.

19. The system of claim 1 wherein the active agent is hydrophilic and has a molecular weight of no greater than about 1200 g/mol.

20. The system of claim 1 wherein the polyurethane is present in the miscible polymer blend in an amount of about 0.1 wt-% to about 99.9 wt-%, based on the total weight of the blend.

21. The system of claim 1 wherein the second polymer is present in the miscible polymer blend in an amount of about 0.1 wt-% to about 99.9 wt-%, based on the total weight of the blend.

22. The system of claim 1 which is in the form of microspheres, beads, rods, fibers, or other shaped objects.

23. The system of claim 22 wherein the critical dimension of the object is no greater than about 10,000 microns.

24. The system of claim 1 which is in the form of a film.

25. The system of claim 24 wherein the thickness of the film is no greater than about 1000 microns.

26. The system of claim 24 wherein the film forms a patch or a coating on a surface.

27. The system of claim 1 wherein the second polymer has at least one Tg lower than at least one Tg of the polyurethane.

28. An active agent delivery system comprising an active agent and a miscible polymer blend comprising a polyurethane and a second polymer, wherein the miscible polymer blend controls the delivery of the active agent, wherein the second polymer is not a hydrophobic cellulose ester, whereindelivery of the active agent occurs predominantly under permeation control.

29. An active agent delivery system comprising an active agent and a miscible polymer blend comprising a polyurethane and a second polymer; wherein:
the second polymer is selected from the group consisting of a polycarbonate, a polysulfone, a polyurethane, a polyphenylene oxide, a polyimide, a polyamide, a polyester, a polyepoxide, and a styrene-acrylonitrile copolymer, a polymethacrylate, a poly(methyl methacrylate), and combinations thereof;
the active agent is hydrophobic and has a molecular weight of no greater than about 1200 g/mol;
the active agent has a solubility parameter, the polyurethane has a soft segment solubility parameter and a hard segment solubility parameter, and the second polymer has at least one solubility parameter;
the difference between the solubility parameter of the active agent and the solubility parameter of the polyurethane hard segment Is no greater than about 10 J^{1/2}/cm^{3/2}, and/or the difference between the solubility parameter of the active agent and the solubility parameter of the polyurethane soft segment Is no greater than about 10 J^{1/2}/cm^{3/2}, and the difference between the solubility parameter of the active agent and at least one solubility parameter of the second polymer is no greater than about 10 J^{1/2}/cm^{3/2}; and
the difference between the solubility parameter of the polyurethane hard segment and at least one solubility parameter of the second polymer is no greater than about 5 J^{1/2}/cm^{3/2}, and/or the difference between the solubility parameter of the polyurethane soft segment and at least one solubility parameter of the second polymer is no greater than about 5 J^{1/2}/cm^{3/2}.

30. The system of claim 29 wherein the second polymer has at least one Tg equal to or higher than all Tg's of the polyurethane.

31. An active agent delivery system comprising:
a miscible polymer blend comprising a first hydrophobic polymer selected from the group consisting of a poly(carbonate urethane) and a poly(ether urethane), and a second hydrophobic polymer selected from the group consisting of a polycarbonate, a poly(ether urethane), and a poly(carbonate urethane); wherein the first polymer has a hard phase Tg of about 20° C. to about 60° C. and the second polymer has at least one Tg of about 80° C. to about 150° C.; and
an active agent incorporated in the miscible polymer blend, wherein the active agent is hydrophobic and has a molecular weight of no greater than about 1200 g/mol.

32. An active agent reservoir delivery system comprising:
a base coat comprising a hydrophilic polymer and an active agent incorporated therein, wherein the active agent is hydrophilic and has a molecular weight of no greater than about 1200 g/mol; and
a cap coat comprising a miscible polymer blend comprising a first hydrophobic poly(ether urethane) having a hard phase Tg of about 20° C. to about 60° C. and a second hydrophobic poly(ether urethane) having a hard phase Tg of about 80° C. to about 150° C.

33. A medical device comprising the active agent delivery system of claim 1.

34. A medical device comprising the active agent delivery system of claim 28.

35. A medical device comprising the active agent delivery system of claim 29.

36. A medical device comprising the active agent delivery system of claim 31.

37. A medical device comprising the active agent delivery system of claim 32.

38. A medical device comprising:
a substrate surface;
a polymeric undercoat layer adhered to the substrate surface; and
a polymeric top coat layer adhered to the polymeric undercoat layer; wherein the polymeric top coat layer comprises an active agent incorporated within a miscible polymer blend comprising a polyurethane and a second polymer, wherein the second polymer is not a hydrophobic cellulose ester.

39. The medical device of claim 38 wherein the second polymer is selected from the group consisting of a polycarbonate, a polysulfone, a polyurethane, a polyphenylene oxide, a polyimide, a polyamide, a polyester, a polyepoxide, a styrene-acrylonitrile copolymer, polymethacrylate, a poly(methyl methacrylate), and combinations thereof.

40. The medical device of claim 38 wherein the polymer undercoat layer comprises a polyurethane.

41. The medical device of claim 38 which is an implantable device.

42. The medical device of claim 38 which is an extracorporeal device.

43. The medical device of claim 38 selected from the group consisting of a stent, stent graft, anastomotic connector, lead, needle, guide wire, catheter, sensor, surgical instrument, angioplasty balloon, wound drain, shunt, tubing, urethral insert, pellet, implant, blood oxygenator, pump, vascular graft, valve, pacemaker, orthopedic device, replacement device for nucleus pulposus, and intraocular lense.

44. The medical device of claim 38 wherein the active agent is hydrophobic and has a molecular weight of no greater than about 1200 g/mol.

45. The medical device of claim 38 wherein the active agent is hydrophilic and has a molecular weight of no greater than about 1200 g/mol.

46. The medical device of claim 38 wherein the second polymer has at least one Tg equal to or higher than all Tg's of the polyurethane.

47. The medical device of claim 38 wherein the second polymer has at least one Tg lower than at least one Tg of the polyurethane.

48. The medical device of claim 38 wherein delivery of the active agent occurs predominantly under permeation control.

49. A stent comprising:
a substrate surface;
a polymeric undercoat layer adhered to the substrate surface; and
a polymeric top coat layer adhered to the undercoat layer;
wherein the polymeric top coat layer comprises an active agent incorporated within a miscible polymer blend comprising a polyurethane and a second polymer having at least one Tg equal to or higher than all Tg's of the polyurethane; wherein the second polymer is selected from the group consisting of a polycarbonate, a polysulfone, a polyurethane, a polyphenylene oxide, a polyimide, a polyamide, a polyester, a polyepoxide, a styrene-acrylonitrile copolymer, a polymethacrylate, a poly(methyl methacrylate), and combinations thereof.

50. The stent of claim 49 wherein the active agent is hydrophobic and has a molecular weight of no greater than about 1200 g/mol.

51. A stent comprising:
a substrate surface;
a polymeric undercoat layer adhered to the substrate surface; and
a polymeric top coat layer adhered to the undercoat layer;
wherein the polymeric top coat layer comprises an active agent incorporated within a miscible polymer blend comprising a polyurethane and a second polymer having at least one Tg lower than at least one Tg of the polyurethane.

52. A stent comprising:
a substrate surface;
a polymeric undercoat layer adhered to the substrate surface;
a base coat adhered to the undercoat layer, wherein the base coat comprises a hydrophilic polymer and a hydrophilic active agent; and
a cap coat adhered to the base coat, wherein the cap coat comprises a miscible polymer blend comprising a first hydrophobic poly(ether urethane) having a hard phase Tg of about 20° C. to about 60° C. and a second hydrophobic poly(ether urethane) having a hard phase Tg of about 80° C. to about 150° C.

53. A method for delivering an active agent to a subject subject, the method comprising:
providing an active agent delivery system comprising an active agent and a miscible polymer blend comprising a polyurethane and a second polymer, wherein the miscible polymer blend controls the delivery of the active agent, and further wherein the second polymer is not a hydrophobic cellulose ester; and
contacting the active agent delivery system with a bodily fluid, organ, or tissue of a subject subject.

54. The method of claim 53 wherein the second polymer has at least one Tg equal to or higher than all Tg's of the polyurethane.

55. The method of claim 53 wherein the second polymer is selected from the group consisting of a polycarbonate, a polysulfone, a polyurethane, a polyphenylene oxide, a polyimide, a polyamide, a polyester, a polyepoxide, a styrene-acrylonitrile copolymer, a polymethacrylate, a poly(methyl methacrylate), and combinations thereof.

56. The method of claim 53 wherein the active agent is incorporated within the miscible polymer blend.

57. The method of claim 53 wherein the active agent is incorporated within an inner matrix and the miscible polymer blend initially provides a barrier to permeation of the active agent.

58. The method of claim 53 wherein the active agent is hydrophobic and has a molecular weight of no greater than about 1200 g/mol.

59. The method of claim 53 wherein the active agent is hydrophilic and has a molecular weight of no greater than about 1200 g/mol.

60. The method of claim 53 wherein the second polymer has at least one Tg lower than at least one Tg of the polyurethane.

61. A method of forming an active agent delivery system comprising:
combining a polyurethane and a second polymer to form a miscible polymer blend, wherein the second polymer is not a hydrophobic cellulose ester; and
combining at least one active agent with the miscible polymer blend such the miscible polymer blend controls the delivery of the active agent.

62. The method of claim 61 wherein the second polymer has at least one Tg equal to or higher than all Tg's of the polyurethane.

63. The method of claim 61 wherein the second polymer is selected from the group consisting of a polycarbonate, a polysulfone, a polyurethane, a polyphenylene oxide, a polyimide, a polyamide, a polyester, a polyepoxide, a styrene-acrylonitrile copolymer, a polymethacrylate, a poly(methyl methacrylate), and combinations thereof.

64. The method of claim 61 wherein the active agent is incorporated within the miscible polymer blend.

65. The method of claim 61 wherein the active agent is incorporated within an inner matrix and the miscible polymer blend initially provides a barrier to permeation of the active agent.

66. The method of claim 61 wherein the active agent is hydrophobic and has a molecular weight of no greater than about 1200 g/mol.

67. The method of claim 61 wherein the active agent is hydrophilic and has a molecular weight of no greater than about 1200 g/mol.

68. The method of claim 67 wherein combining at least one active agent with the miscible polymer blend comprises combining the hydrophilic active agent with a hydrophilic polymer and forming an inner matrix of a reservoir system with the miscible polymer blend forming a cap coat.

69. The method of claim 68 wherein delivery of the active agent occurs predominantly under permeation control.
